(19) United States
(12) Patent Application Publication
(10) Pub. No.: US 2020/0253504 A1
(43) Pub. Date: Aug. 13, 2020

(54) SYSTEMS AND METHODS FOR TISSUE CHARACTERIZATION

(71) Applicants: Daniel Shen, Palo Alto, CA (US); Brett Herscher, Cupertino, CA (US); David Krawzsenek, Mountain View, CA (US); Kyler Connelly, Foster City, CA (US); Michael Baker, Arlington, MA (US); R. Maxwell Flaherty, Topsfield, MA (US); J. Christopher Flaherty, Auburndale, FL (US)

(72) Inventors: Daniel Shen, Palo Alto, CA (US); Brett Herscher, Cupertino, CA (US); David Krawzsenek, Mountain View, CA (US); Kyler Connelly, Foster City, CA (US); Michael Baker, Arlington, MA (US); R. Maxwell Flaherty, Topsfield, MA (US); J. Christopher Flaherty, Auburndale, FL (US)

(21) Appl. No.: 16/758,494

(22) PCT Filed: Nov. 1, 2017
(86) PCT No.: PCT/US2018/058701

§ 371 Date: Apr. 23, 2020

Related U.S. Application Data
(60) Provisional application No. 62/580,017, filed on Nov. 1, 2017.

(51) Int. Cl. A61B 5/053; A61B 5/01; A61B 5/00

(52) U.S. Cl. CPC A61B 5/0537; A61B 5/0538; A61B 5/1032; A61B 5/7267; A61B 5/01

(57) ABSTRACT

Systems for characterizing tissue comprise: a tissue probe comprising a plurality of probe electrodes; a signal generator, a controller, and a user interface. The signal generator is in communication with the probe electrodes for delivering a drive signal to at least one probe electrode. The controller receives a recorded signal from one or more probe electrodes, the recorded signal resulting from delivery of the drive signal through tissue proximate the plurality of probe electrodes. The controller is configured to determine patient-to-patient differences when the tissue probe is proximate a known type of patient tissue and/or bodily fluid, and it produces tissue characterization information based on the recorded signal and the patient-to-patient differences. The user interface provides the tissue characterization information to an operator of the system. Methods of characterizing tissue are also provided.

### RELATED APPLICATIONS

This application is related to: U.S. Provisional Patent Application Ser. No. 62/580,017, filed Nov. 1, 2017, titled “Surgical Methods and Systems for Tissue Identification”; the content of which is incorporated herein by reference in its entirety for all purposes.

### FIELD OF THE INVENTION

The present invention relates generally to medical devices and methods. More specifically, the invention relates to systems and methods for identification and/or other characterization of tissue.

### BACKGROUND

Significant time is spent before and during surgical and other clinical procedures to properly identify tissues which are to be treated, cut or removed. Even with open surgical procedures, some tissues can be difficult to differentiate. When a minimally invasive surgical technique is used, it can become even more difficult to identify and differentiate between different tissue types. Significant surgical complications can arise from improper identification of tissue or misidentification of tissue not intended to be treated or cut. Cutting of unintended tissue can happen when the tissue is difficult to differentiate, when it is obscured by other tissues, fluid, or materials, or when it is in an unanticipated anatomical location or takes an unanticipated form or visual appearance.

Searching for or working around nerves, vessels, glands, ducts, lumens or other structures during a clinical procedure by detailed dissection and careful separation of tissues can consume valuable staff and operating room time. This process relies heavily on clinician skill, experience, and the desired or required speed of the clinical procedure. However, significant person to person variation exists that affects the ability and efficiency in properly identifying or locating these sensitive structures.

Therefore, a need exists for improved tissue identification and differentiation systems and methods for improving the efficiency of procedures and reducing complications.

### SUMMARY

According to one aspect of the present inventive concepts, a system for characterizing tissue comprising: a tissue probe comprising a plurality of probe electrodes; a signal generator in communication with the probe electrodes for delivering a drive signal to at least one probe electrode; a controller for receiving a recorded signal from one or more probe electrodes, the recorded signal resulting from delivery of the drive signal through tissue proximate the plurality of probe electrodes. The controller is configured to determine patient-to-patient differences when the tissue probe is proximate a known type of patient tissue and/or bodily fluid, and it produces tissue characterization information based on the recorded signal and the patient-to-patient differences. The system further comprises a user interface for providing the tissue characterization information to an operator of the system.

In some embodiments, the tissue probe includes electrodes for cutting and/or coagulating tissue by selective application of energy. A probe electrode can deliver the energy to cut and/or coagulate the tissue.

In some embodiments, the tissue probe comprises a coating positioned over the at least one probe electrode to form a non-contact probe electrode.

In some embodiments, the one or more probe electrodes comprise the at least one probe electrode.

In some embodiments, the at least one electrode and/or the one or more electrodes comprise one or more electrodes each having a maximum dimension of 2 mm.

In some embodiments, the tissue probe comprises a removable tip, and the removable tip comprises the at least one electrode and/or the one or more electrodes.

In some embodiments, the drive signal is used by the controller for determining tissue type, and the controller is configured to provide energy to cut and/or coagulate tissue based on the determined tissue type.

In some embodiments, the at least one electrode is in contact with tissue during the delivery of the drive signal and/or the one or more electrodes are in contact with tissue during the recording of the recorded signal.

In some embodiments, the signal generator comprises a swept frequency generator.

In some embodiments, the signal generator delivers the drive signal at an energy level of no more than 10 mWatts and/or no more than 100 Watts.

In some embodiments, the controller is configured to utilize machine learning and/or a neural net to determine the tissue characterization information.

In some embodiments, the tissue characterization information is based on a patient parameter selected from the group consisting of: gender; age; race; ethnicity; body mass index (BMI); body composition; one or more medical conditions; hydration level; temperature; electrolyte concentration; weight; a biological characteristic; and combinations thereof.

In some embodiments, the tissue characterization information is based on a patient environment parameter selected from the group consisting of: temperature; humidity; pressure; environmental electromagnetic radiation; elevation; location; and combinations thereof.

In some embodiments, the controller is configured to identify multiple types of tissue.

In some embodiments, the controller is configured to produce the tissue characterization information within 5 seconds, within 2 seconds, and/or within 50 milliseconds of the recording of the recorded signals.

In some embodiments, the recorded signal represents information of tissue that is offset from the one or more electrodes.

In some embodiments, the recorded signal represents information of tissue that is in contact with the one or more electrodes.

In some embodiments, the at least one electrode and/or the one or more electrodes are configured to be placed into contact with and/or in close proximity to at least two types of patient tissue for calibration of the system.

In some embodiments, the controller is configured to calibrate the system for patient-to-patient differences in tissue impedance of the same tissue type.

In some embodiments, the controller is configured to adjust the tissue characterization information based on an initial patient reference baseline of two or more of patient muscle, fat, or blood.

In some embodiments, the user interface provides one or more of an audible signal, a light, a vibration, a numerical value related to the proximity to the identified tissue, or a notification of a type of tissue in contact with and/or in close proximity to the at least one electrode and/or the one or more electrodes.

In some embodiments, the tissue characterization information comprises tissue type information.

According to another aspect of the present inventive concepts, a system for characterizing tissue comprising: a tissue probe comprising a plurality of tissue-contacting electrodes; a signal generator comprising a swept and/or stepped frequency generator in communication with the electrodes for delivering a drive signal comprising a plurality of different frequencies to one of the plurality of electrodes; a controller receiving a recorded signal resulting from delivery of the drive signal through tissue proximate the plurality of electrodes, the controller producing tissue characterization information based on the recorded signal; and a user interface providing the tissue characterization information to an operator of the system.

According to another aspect of the present inventive concepts, a system for determining an insertion depth of a needle into tissue comprising: a needle comprising a plurality of tissue-contacting electrodes; a signal generator comprising a swept and/or stepped frequency generator in communication with the electrodes for delivering a drive signal comprising a plurality of different frequencies to one of the plurality of electrodes; a controller receiving a recorded signal resulting from delivery of the drive signal through tissue proximate the plurality of electrodes, the controller producing tissue characterization information based on the recorded signal; and a user interface providing information about a location of the needle within the tissue and/or a warning when the needle has exited the tissue.

In some embodiments, the controller measures a change in the signal as the needle is advanced into a layer of tissue and the user interface provides information indicating when the needle is nearing an edge of the layer of tissue. The layer of tissue can be a wall of the heart.

According to another aspect of the present inventive concepts, a method for characterizing tissue during a clinical procedure, the method comprising: providing a surgical instrument comprising a tissue probe having a plurality of tissue-contacting electrodes, a signal generator comprising a swept and/or stepped frequency generator in communication with the electrodes for delivering a drive signal comprising a plurality of different frequencies to one of the plurality of electrodes, and a controller receiving a recorded signal resulting from delivery of the drive signal through tissue proximate the plurality of electrodes, the controller producing tissue characterization information based on the recorded signal; inserting the surgical instrument into a surgical site and contacting the tissue probe to tissue during the clinical procedure; and providing tissue characterization information comprising tissue type information, prior to and/or during the cutting and/or other treating of tissue.

In some embodiments, the method further comprises a step of determining a patient baseline reference for adjustment of the surgical instrument for patient-to-patient differences by contacting the tissue probe with a known type of patient tissue and/or bodily fluid prior to insertion of the surgical instrument.

In some embodiments, the determining patient baseline reference step adjusts for patient-to-patient differences in tissue impedance of the same tissue type.

In some embodiments, the determining patient baseline reference step adjusts the output of the surgical instrument based on an initial patient reference of two or more of patient muscle, fat or blood.

In some embodiments, the determining patient baseline reference step involves placing the electrodes into contact with and/or in close proximity to at least two types of patient tissue and adjusting the surgical instrument.

In some embodiments, the tissue-contacting electrodes each have a maximum dimension of 2 mm. The tissue probe can comprise a removable tip, and the removable tip can comprise the tissue-contacting electrodes.

In some embodiments, the tissue-contacting electrodes provide recorded signals to the controller for determining tissue type and the tissue-contacting electrodes selectively deliver treatment energy to cut and/or coagulate tissue.

In some embodiments, the tissue probe includes electrodes for cutting and/or coagulating tissue by selective application of energy

In some embodiments, the tissue characterization information includes one or more of an audible signal, a light, a vibration, a numerical value related to the proximity to the identified tissue, or a notification of a type of tissue in contact with and/or in close proximity to the tissue probe.

According to another aspect of the present inventive concepts, a method of reducing complications in a clinical procedure, the method comprising: providing a surgical instrument comprising a tissue probe having a plurality of electrodes positioned in a tip portion of the tissue probe; providing a signal generator in communication with at least one of the electrodes; delivering a drive signal through tissue proximate the probe tip; providing an indication of tissue type and/or other tissue characterization information based on tissue impedance determined by delivering the drive signal; and communicating information regarding the tissue type and/or the other tissue characterization information to an operator and/or to the surgical instrument to prevent undesired cutting and/or other undesired treating of specific tissue, and/or to selectively cut and/or otherwise treat specific desired tissue.

In some embodiments, the clinical procedure is a minimally invasive surgery. The clinical procedure can be a facial procedure and the tissue type identified can be a nerve.

According to another aspect of the present inventive concepts, a method of reducing clinical procedure time, the method comprising: providing a surgical instrument comprising a tissue probe having a plurality of electrodes positioned in a tip portion of the surgical instrument; providing a signal generator in communication with at least one of the electrodes; delivering a drive signal through tissue adjacent the surgical instrument tip portion; providing an indication of tissue type and/or other tissue characterization information based on tissue impedance determined by delivering the drive signal; and communicating information regarding the tissue type and/or other tissue characterization information to an operator and/or to the surgical instrument to prevent undesired cutting and/or other undesired treating of specific tissue, and/or to selectively cut and/or otherwise treat specific desired tissue.

In some embodiments, the surgical instrument includes a tissue cutting portion adjacent the plurality of electrodes to allow identification of tissue type prior to cutting of the tissue.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a schematic view of one embodiment of a system for classification of tissue, consistent with the present inventive concepts.

FIG. 2 illustrates a schematic view of another embodiment of a system for classification of tissue, consistent with the present inventive concepts.

FIG. 3 illustrates a perspective view of a probe tip with an electrode array, consistent with the present inventive concepts.

FIG. 3A illustrates a perspective view of a probe tip with a covered electrode array, consistent with the present inventive concepts.

FIG. 4 illustrates a front view of one embodiment of a user interface, consistent with the present inventive concepts.

FIG. 5 illustrates a schematic cross section of a probe tip having a plurality of electrodes contacting tissue, consistent with the present inventive concepts.

FIG. 6 illustrates a time-domain representation of a waveform comprising a time-interleaved drive signal and treatment energy signal, consistent with the present inventive concepts.

FIG. 7 illustrates a schematic view of another embodiment of a system for classification of tissue having a probe further configured as a monopolar treatment energy delivery device, consistent with the present inventive concepts.

FIG. 8 illustrates a schematic view of another embodiment of a system for classification of tissue having a probe further configured as a bipolar treatment energy delivery device, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereo” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the terms “treatment”, “treatment procedure”, and their derivatives refer to medical procedures in which a patient is being treated, such as when target tissue of the patient is being treated. Treatment procedures include but are not limited to: delivery of energy to tissue; ablating tissue; cutting, dissecting, and/or removing tissue; causing necrosis or otherwise denaturing of tissue; delivery of a drug or other agent to tissue; delivery of radiation and/or a radioactive substance to tissue; and combinations of these.

As used herein, the term “target tissue” refers to tissue to which a treatment procedure is intended to be performed.

As used herein, the term “non-target tissue” refers to tissue that is to be avoided (e.g. minimally affected) during a treatment procedure performed on target tissue.

As used herein, the term “neighboring tissue” comprises tissue proximate one or more electrodes. Neighboring tissue can comprise tissue in contact with the electrodes, and/or tissue offset but in relative proximity to the electrodes.

As used herein, the term “drive signals” comprises one or more signals delivered by the electrodes to neighboring tissue, the drive signals used to characterize the neighboring tissue.

As used herein, the term “recorded signals” comprises one or more signals recorded by electrodes, the recorded signals used to produce tissue characterization information of the present inventive concepts.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Provided herein are systems for identification, differentiation, and/or other characterization of tissue. The systems can include a probe having a plurality of tissue-contacting probe electrodes, such as for delivering “drive signals” to tissue, drive signals 401 shown. Drive signals 401 can include signals comprising energy that is delivered to tissue in a bipolar and/or other multipolar (“bipolar” herein) manner. A signal generator is in communication with (e.g. electrically connected to) the probe electrodes, for selectively applying the drive signals 401 to the probe electrodes, such as when these probe electrodes are positioned proximate a location in which a surgical or other clinical procedure is to be performed (“surgical site” herein), such as when the probe electrodes are in contact with and/or at least proximate to “neighboring tissue” to be characterized. A processor receives “recorded signals”, recorded signals 402 shown (e.g. recorded electromagnetic waves and/or other signals), from the probe electrodes, the recorded signals 402 resulting from delivery of the drive signals 401 through neighboring tissue. The processor analyzes the recorded signals 402 (e.g. compares the recorded signals 402 to the delivered drive signals 401), the analysis resulting in identifying, differentiating, and/or otherwise characterizing the neighboring tissue (e.g. tissue in contact with the electrodes and/or tissue in relative proximity to the electrodes). For example, the processor can measure or otherwise analyze the properties of the recorded signals 402, the recorded signals 402 representing energy that is reflected, transmitted, and/or conducted from the neighboring tissue that is a result of the delivery of the drive signals 401 to the neighboring tissue. These recorded signal 402 properties can include magnitude, phase, and/or delay (e.g. of an electromagnetic wave or other signal, “electromagnetic wave” or “signal” herein). These recorded signal 402 properties are dependent upon the reflective, absorptive, conductive, reactive, and/or transmissive properties of the neighboring tissue, and thus can be used to characterize the neighboring tissue (e.g. when compared to similar data for known tissue types). A feedback indicator or other user interface can be included to provide information about the determined tissue characteristics of the neighboring tissue (e.g. tissue at the tip of the probe) to an operator of the system (e.g. a surgeon or other clinician), such as for use in making decisions during the performance of the clinical procedure. With this information about tissue type and/or other tissue characteristics provided by the system, the clinician can make safer and faster decisions during the clinical procedure. The systems of the present inventive concepts can both reduce procedure time and reduce procedural complications. The information about tissue type and/or other tissue characteristics provided by the system can also be used to inform (e.g. provide information to) algorithms, tools, machines, and/or other devices, and can result in automated changes to treatment, such as to avoid undesired cutting or other undesired changes to non-target tissue, as described herebelow.

The processor and/or other system component can include a patient tissue reference module, such as for adjusting for patient-to-patient differences (“PtoP differences” herein) in tissue properties. For example, a hydrated patient and a dehydrated patient can exhibit significantly different electrical properties of their tissues. The patient tissue reference module of the present inventive concepts can compensate for these and/or other differences, which can result in improved accuracy and/or sensitivity in tissue characterization measurements. The patient tissue reference module can be used for adjusting the output of the system processor (e.g. adjust the tissue characterization information provided) to accommodate for: PtoP differences; patient tissue changes over time; or both. A patient tissue reference module can be set (e.g. initiated, adjusted, and/or calibrated) by contacting the probe electrodes with one or more known types of tissue and/or bodily fluids (e.g. of the particular patient to be treated or otherwise) prior to use of the system (e.g. use that includes providing tissue characterization information). The system can then be adjusted or otherwise calibrated based on feedback from the patient tissue reference module. Alternatively or additionally, the patient tissue reference module can be set by taking measurements from blood samples, and/or by taking other measurements from the patient that can indicate characteristics of the patient at the time of a clinical procedure, such as characteristics related to hydration, tissue type composition, and/or electrolyte concentration.

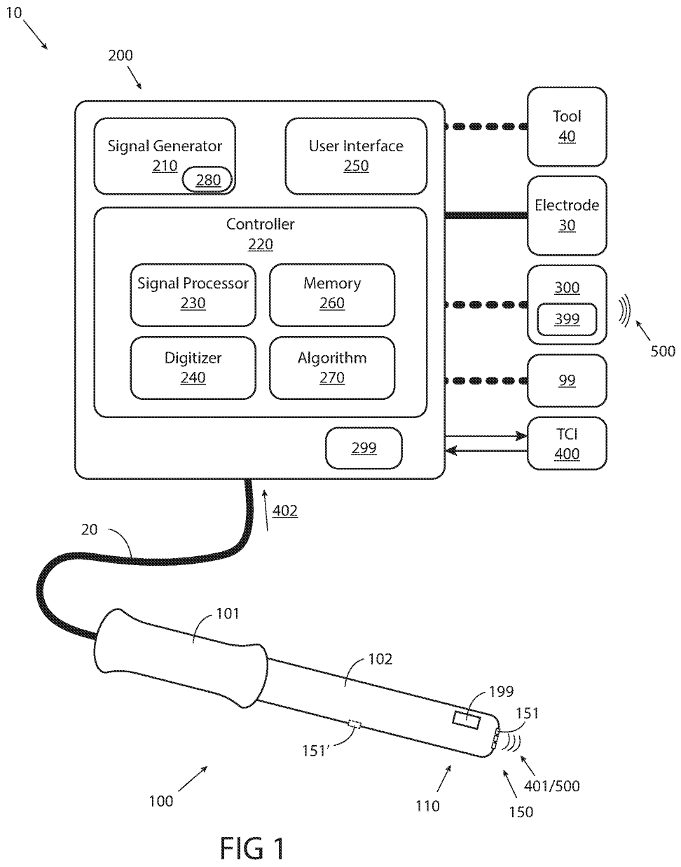

Referring now to FIG. 1, a system for characterizing tissue is illustrated, consistent with the present inventive concepts. System 10 includes a tissue probe for contacting tissue to be characterized, probe 100. Probe 100 includes an electrode assembly 150 comprising one or more probe electrodes 151. Probe 100 is configured to operably attach to console 200, which provides signals (e.g. drive signals 401) and/or other energy to probe 100, and receives signals (e.g. recorded signals 402) from probe 100. Drive signals 401 and recorded signals 402 can be used by console 200 to characterize tissue. Probe 100 can operably attach to console 200 via one or more conduits, such as cable 20 shown. Console 200 analyzes, performs one or more calculations upon, and/or otherwise processes the recorded signals 402 received to produce tissue characterization information, TCI 400 shown.

System 10 can be used by a surgeon, clinician, and/or other operator to identify or otherwise characterize tissue, such as target tissue intended to receive a treatment and/or non-target tissue to avoid being treated (e.g. avoid being undesirably affected). System 10 can include a device configured to perform a treatment (e.g. a tissue treatment), such as treatment device 300 described herebelow, or a probe 100 that is configured to both characterize tissue as well as perform a treatment. System 10 can be used to characterize various types of tissues, in various clinical procedures, such as is described herebelow.

Probe 100 is configured to record information related to characteristics of tissue, via the recorded signals 402 received by console 200. Probe 100 comprises handle 101, shaft 102, and a distal portion, tip 110. Probe 100 can comprise one or more probes 100, such as when system 10 includes multiple similar and/or dissimilar probes 100. In some embodiments, system 10 includes multiple probes 100 that have a dissimilar construction selected from the group consisting of: different type of a probe electrode 151; different size of a probe electrode 151; different tissue-contacting surface area of a probe electrode 151; different arrangement of multiple probe electrodes 151; different shape of handle 101; different tip 110 and/or shaft 102 length, width, and/or other geometric difference; and combinations thereof.

In some embodiments, tip 110 and/or another portion of probe 100 can be articulated, either manually by an operator or as controlled by console 200. Cable 20 can include one or more flexible linkages that operably connect to corresponding flexible linkages within probe 100. These linkages can be manipulated by controller 220 to articulate tip 110. In some embodiments, tip 110 and/or another portion of probe 100 is robotically controlled by console 200, such as a robotic control that is at least partially based on TCI 400 (e.g. robotic control in which console 200 (e.g. algorithm 270 described herebelow) analyzes TCI 400 and performs the robotic control based on the analysis (e.g. in a closed-loop fashion)).

Tip 110 comprises the distal portion of probe 100 (e.g. a distal portion of shaft 102), and one or more probe electrodes 151 can be positioned on and/or within tip 110. Tip 110 can be rigid, flexible, or include both rigid and flexible portions.

Electrode assembly 150 can comprise an array or other arrangement of one or more probe electrodes 151. Probe electrodes 151 comprises one or more electrodes that are at least configured to record electrical signals (e.g. recorded signals 402) that can be used to produce TCI 400. In some embodiments, one or more probe electrodes 151 are further configured to deliver energy (e.g. deliver drive signals 401 and/or deliver therapeutic energy) and/or perform another function.

Probe electrodes 151 can be positioned on and/or within tip 110, on and/or within shaft 102, and/or on and/or within another portion of probe 100. In some embodiments, probe electrodes 151 comprise an electrode 151′ positioned on shaft 102, proximal to tip 110 (as shown). In some embodiments, probe electrodes 151 comprise one or more electrodes positioned on and/or within tip 110 and one or more electrodes positioned on and/or within shaft 102.

The shape, size and/or spacing of probe electrodes 151 can be selected to provide recorded signals 402 representing localized tissue characterization, and can vary depending on the type of tissue being characterized. The size and/or spacing of the probe electrodes 151 determines the zone (e.g. volume) of tissue through which the delivered energy (drive signals 401) will be transmitted, and thus controls the area or volume of tissue sensed (e.g. the “neighboring tissue” being characterized). For example, when locating small tissue structures such as nerves, a close spacing and small size of probe electrodes 151 provides more precise location of the structures (e.g. more precise differentiation of nerve tissue such as non-nerve tissue both present in neighboring tissue). Size and spacing of probe electrodes 151 can be somewhat larger for characterizing (e.g. differentiating) larger structures, such as skin, fat, and/or certain muscular structures. Microscale or nanoscale sized probes 100 can be provided for particularly small surgeries, such as eye surgery.

In some embodiments, all or at least a subset of probe electrodes 151 are of relatively equal size, such as all or a subset of probe electrodes 151 with a diameter (e.g. an equivalent diameter) and/or width (e.g. minor axis length) of between 0.0001 mm and 30 mm, such as between 0.001 mm and 4 mm, such as between 0.1 mm and 1.5 mm. In some embodiments, all or a subset of probe electrodes 151 can comprise a diameter of at least 0.1 μm, such as a diameter less than 20 μm. In embodiments with relatively small probe electrodes 151 and/or small probe electrode assemblies 150, probe 100 can include one or more flexible printed circuit boards to form the probe electrodes 151, and/or electrodes formed using lithographic techniques. Probe electrodes 151 formed using these manufacturing methods can have a size (e.g. diameter) of at least 10 nm and/or no more than 500 μm, such as between 10 μm and 200 μm. U.S. Pat. No. 6,024,702 describes one example of the use of printed circuit technology for forming implantable electrodes for monitoring tissue electrical activity, the content of which is incorporated herein by reference for all purposes.

Spacing between two or more probe electrodes 151 can be about one twentieth to about ten times the probe electrode 151 diameter or width, and in some embodiments, probe electrode 151 spacing is between about one half to about five times the probe electrode 151 diameter and/or width. All or a subset of probe electrodes 151 can have a length of no more than about ten times their width. In some embodiments, all or a subset of probe electrodes 151 have a substantially circular and/or a substantially rectangular profile (e.g. the profile that is to contact with and/or is otherwise in relatively close proximity to the tissue to be characterized).

Probe electrodes 151 can comprise of one or more of materials, such as one or more materials selected from the group consisting of: gold; gold plated copper; platinum iridium; platinum; steel; nickel; titanium; copper, such as copper positioned on polyimide; and combinations of these. Probe 100, such as tip 110, can comprise (e.g. be formed of) an electrical insulating material, such as a polymer. Tip 110 and/or probe electrodes 151 can be rigid, flexible, or include both rigid and flexible portions. In some embodiments, probe electrode 151 spacing is relatively fixed (e.g. constructed and arranged to limit variations in spacing during use or otherwise).

As described hereabove, probe electrodes 151 can comprise two or more electrodes configured to deliver energy (e.g. drive signals 401 and/or treatment energy 500) to tissue in a bipolar and/or other multipolar mode. Alternatively, probe electrode 151 can comprise one or more electrodes that transmit signals to a separate electrode (e.g. a return electrode), electrode 30 shown, in a monopolar fashion. For example, electrode 30 can be placed under the patient during surgery. Alternatively or additionally, electrode 30 can be placed at a location proximate the surgical site, such as a location underneath an organ or other tissue being treated, or at other locations within or outside of the surgical site (e.g. but proximate the surgical site).

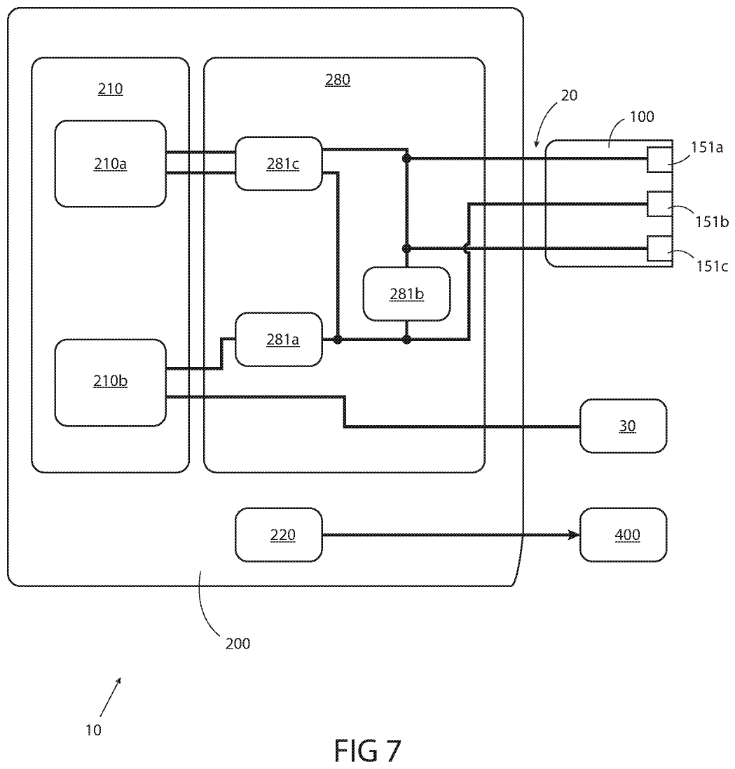

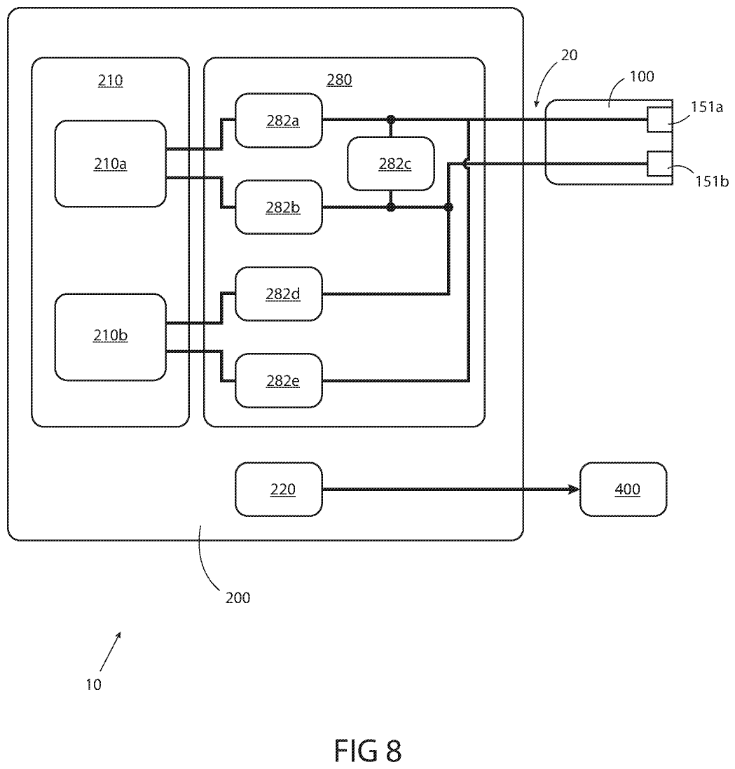

As described herein, probe electrodes 151 can receive bipolar and/or other multipolar signals from a signal generator 210 (e.g. bipolar and/or other multipolar drive signals 401 and/or treatment energy 500). In some embodiments, probe electrodes 151 comprise a first set of electrodes 151a configured to receive multipolar signals to produce TCI 400 comprising characterization of tissue in proximity to the first set of electrodes 151a. The same first set of electrodes 151a can be configured to deliver treatment energy 500 (e.g. in a monopolar and/or multipolar mode), such as to cut, ablate, heat, cool, deliver energy to, and/or otherwise treat tissue in proximity to the first set of electrodes 151a using TCI 400 (e.g. to avoid cutting non-target tissue). The delivery to electrodes 151a of both the drive signals 401 and the treatment energy 500 can be accomplished by signal generator 210 via an electrically switchable isolation network (e.g. isolation network 280 described herebelow and in reference to FIGS. 7 and 8) which electrically separates electrodes 151a during delivery of drive signals 401 (e.g. during tissue characterization), and connects electrodes 151a together during treatment energy 500 delivery (e.g. to maximize electrode surface area delivering energy).

Challenges of transmitting therapy and treatment over a thin and flexible cable (e.g. cable 20) can be addressed by a method of multiplexing both of drive signal 401 and treatment energy 500 through a single impedance-controlled electrical connection. System 10 can include active and/or passive switch networks in either console 200, cable 20, and/or probe 100, such as to achieve multiplexing of treatment energy 500 and drive signal 401 delivery, while protecting the patient, components of system 10, and its operator(s) from high-voltage waveforms. Mitigation of harmonic content from using a nonlinear network (e.g. isolation network 280 described herein) can be addressed with a filter network that comprises passive electrical filter elements that can be positioned in console 200, cable 20, and/or probe 100.

In some embodiments, all or a subset of probe electrodes 151 comprise a size (e.g. a diameter, a major axis, and/or a minor axis) of at least 0.1 μm, and/or no more than 10 mm. In some embodiments, all or a subset of probe electrodes 151 comprises a maximum dimension of 2 mm (e.g. a diameter, length, and/or major axis that does not exceed 2 mm). Two or more probe electrodes 151 can be positioned with a separation distance of at least 0.1 μm and/or no more than 50mm.

In some embodiments, all or a subset of probe electrodes 151 is covered by a housing and/or a coating, such as coating 152 described herebelow in reference to FIG. 3.

Cable 20 operably attaches probe 100 to console 200. Cable 20 can comprise one or more flexible conduits that include one or more wires or other electrical energy or information carrying conduits (“wires” herein) such as to transfer signals (e.g. drive signals 401) and/or other electrical energy between probe 100 and console 200. In some embodiments, cable 20 comprises one, two, or more components selected from the group consisting of: wires; fluid delivery tubes; mechanical linkages; optical fibers; waveguides; coaxial transmission lines; and combinations of these.

Cable 20 can be pre-attached and/or attachable (via one or more mating connectors) to probe 100. Cable 20 can be pre-attached and/or attachable (via one or more mating connectors) to console 200.

In some embodiments, system 10 comprises one or more electrodes not included in probe 100, such as electrode 30 shown. Electrode 30 can comprise a return electrode, such as a return electrode configured as a return path for drive signals 401 to produce TCI 400 (e.g. signals transmitted by one or more probe electrodes 151), and/or an electrode configured as a return path for electrocautery or other electrical energy delivered to cut, coagulate, ablate, denature, and/or otherwise treat tissue (e.g. as delivered by probe electrodes 151, another electrode of probe 100, and/or an electrode of treatment device 300). In some embodiments, electrode 30 is positioned on the patient's back or leg. Electrode 30 can be attached to console 200 via one or more wires.

In some embodiments, system 10 includes one or more tools, tool 40 shown. Tool 40 can comprise a tool used to perform a calibration of probe 100, console 200, and/or another component of system 10, such as a calibration load as described herebelow. In some embodiments, tool 40 comprises a charging unit, such as a charging unit to recharge a battery and/or capacitor of probe 100 and/or console 200 (e.g. a functional element 199 and/or 299, respectively, comprising a battery and/or capacitor).

In some embodiments, tool 40 comprises an imaging device, such as an MRI, CT Scanner, X-ray, fluoroscope, camera, ultrasound imager, and/or other imaging device. In these embodiments, tool 40 can be used to create a localization map, such as an anatomical map in which TCI 400 is illustrated in reference to a particular location of the patient's anatomy.

Console 200 is configured to receive recorded signals 402 from probe 100, and to produce TCI 400 based on those recorded signals 402. TCI 400 can be provided to a clinician or other operator of system 10, via user interface 250, for example as configured as a feedback indicator. Alternatively or additionally, TCI 400 can be used to control a treatment device (e.g. probe 100 further configured as a treatment device and/or treatment device 300), such as in a closed-loop manner as described herein.

Console 200 can comprise one or more discrete components (e.g. one or more components each surrounded by a housing). In some embodiments, all or a portion of console 200 is positioned within probe 100 (e.g. positioned within housing 101 and/or shaft 102 of probe 100). For example, probe 100 can include one or more electronic assemblies of console 200, such as an electronic assembly including a component (e.g. functional element 299) selected from the group consisting of: a filter (e.g. an electronic filter); a multiplexor; a switching network; an alert element (e.g. a light, display, speaker, and/or vibrational transducer); a waveguide; a load resistance; an analog-to-digital converter; and combinations of these.

In some embodiments, controller 220 or another component of console 200 includes a robotic control module, such as to control a robotic portion of probe 100 and/or treatment device 300, such as robotic control that is at least partially based on TCI 400 as described herein.

Console 200 includes signal generator 210 which provides drives signals 401 to probe electrodes 151 for characterization of tissue. The drive signals 401 provided by signal generator 210 can comprise drive signals including one or more of the following: narrow band signals; wide band signals; time domain signals (e.g. one or more time domain pulses); random signals; pseudorandom signals; periodic signals; a swept sine wave (e.g. “chirp”); a sine wave; a triangle wave; a square wave; a pulse; a pulse train; a combination of signals; and/or a combination of pulses, pulse trains, chirped pulses, sine waves, triangle waves, and/or square waves. In some embodiments, signal generator 210 provides other signals, such as electrocautery or other energy to perform a treatment, treatment energy 500, such as energy delivered to probe 100 (e.g. to probe electrodes 151 and/or functional element 199 comprising one or more electrodes) and/or to treatment device 300.

Signal generator 210 can comprise a stepped and/or a sweep frequency signal generator, such as a generator which generates a continuous sweep of frequencies or stepped series of frequencies over a prescribed frequency range, and in certain cases in a repeated pattern. A swept frequency generator can generate an output (e.g. a drive signal 401) which can be a sinusoidal signal. Such an output can have its frequency automatically varied or swept between two selected frequencies. One complete cycle of the frequency variation is called a sweep. Signal generator 210 can comprise a swept frequency generator configured to provide a swept amplitude of the signal applied to probe electrodes 151.

Examples of signal generators 210 comprising stepped frequency generators include phase locked loop generators and direct synthesizers. While stepped or swept frequency generators can be used, non-stepped signal generation may be used in combination with signal processing to separate the multiple frequencies after transmission through the tissue. For example, console 200 can include a TDANA (time domain automatic network analyzer). A TDANA utilizes time-domain measurements and fast Fourier transform to obtain frequency-domain scattering parameters. Signal generator 210 can be configured to generate one or more specific frequencies or sets of frequencies that can result in enhanced recorded signals 402, such as frequencies that result in increased differentiation in characteristics of TCI 400. In some embodiments, signal generator 210 comprises one or more oscillators, such as a harmonic (e.g. crystal) oscillator and/or a relaxation oscillator.

In some embodiments, signal generator 210 delivers energy in the RF frequency range of from a few Hz to about 20 GHz, such as between 1 kHz and 12 GHz, such as between 10 kHz and 6 GHz. In some embodiments, signal generator 210 provides drive signals 401 at an energy level of at least 0.01 μWatt or at least 1 μWatt and/or no more than 10 mWatts or no more than 100 Watts. Signal generator 210 can also be used to deliver signals at other frequencies, beyond the RF range, including microwave, optical, ultrasound and/or other non-visible forms of energy. For example, signal generator 210 can comprise a microwave signal generator that delivers energy in a frequency from less than 1 MHz and/or to at least 20 GHz. In some embodiments, signal generator 210 delivers signals above approximately 100 kHz, such as when undesired stimulation of nerves is to be avoided.

Signal generator 210 can be configured to vary sweep rates (e.g. of a drive signal 401) from 100 seconds/sweep to 1 microsecond/sweep. Signal generator 210 can be configured to allow an operator to adjust voltage, waveform, power, frequency, phase, duty cycle, duration and/or energy level of a signal, such as an adjustment made depending on the intended application, such as to optimize sensitivity and specificity of the determined TCI 400, and/or to prevent or at least reduce undesired effects on tissue. Alternatively or additionally, one or more components of system 10 (e.g. probe 100, treatment device 300, algorithm 270 and/or another component of console 200, and/or another component of system 10) can be configured to automatically adjusts voltage, waveform, power, frequency, phase, duty cycle, duration and/or energy level of a signal, such as an adjustment made depending on the intended application, such as to optimize sensitivity and specificity of the determined TCI 400, and/or to prevent or at least reduce undesired effects on tissue.

As an alternative, or in addition to a sweeping frequency stimulus, signal generator 210 can be configured to excite tissue by applying a wide-band pulse and/or set of wide-band pulses. These pulses can have zero-mean field energy, and/or non-zero-mean field energy. Signal generator 210 can accomplish this pulse generation using switch networks, avalanche diode techniques, and/or other wide-band generation techniques. The amplitude and duration of the provided signals (e.g. drive signals 401) can be adjusted to accommodate signal integrity concerns, linearity, and receiver considerations. The reflected wave (e.g. recorded signal 402), measured in the time-domain, results and is input into controller 220 for analysis to produce TCI 400.

Signal generator 200 can perform time-interleaving and/or frequency-interleaving of treatment drive signal 401 delivery and treatment energy 500 delivery, such as at a rate sufficient to produce the perception for the operator, that both are happening relatively simultaneously and seamlessly (e.g. as described herebelow in reference to FIGS. 7 and 8). A treatment energy 500 waveform, which could be an electrosurgical cutting electrical signal at high-voltage and frequencies in the range of 200 kHz to 3.3 MHz, is interleaved with drive signal 401, which can be a low-energy signal at higher frequencies, such as frequencies up to 20 GHz. The alternation between these two modes can be performed in a relatively short time period, such as a time period in the range of milliseconds, and it can provide the operator continuous information (e.g. TCI 400) for the tissue proximate electrodes 151. This interleaving mode improves clinician ease-of-use by eliminating the need to turn the tissue characterization mode on and off, with a perceived “always-on” tissue characterization mode. This interleaving mode can be used with monopolar and/or multipolar signal delivery (e.g. monopolar and/or multipolar drive signals 401 and/or treatment energy 500).

In some embodiments, signal generator 210, as an alternative to providing a sweeping frequency stimulus, delivers a wide-band pulse, delivers a collection of frequencies (e.g. a sparse collection of frequencies), and/or delivers a set of pulses as one or more drive signals 401 to excite tissue. These pulses can have zero-mean field energy, or non-zero-mean field energy. Signal generator 210 can provide these pulses using switch networks, avalanche diode techniques, and/or another wide-band generation technique. The amplitude and duration of the pulses can be adjusted to accommodate signal integrity concerns, linearity, and receiver considerations. Console 200 receives, via the probe electrodes 151, the reflected wave(s) (recorded signal 402), which can be measured in the time-domain and used to characterize tissue (e.g. used to produce TCI 400).

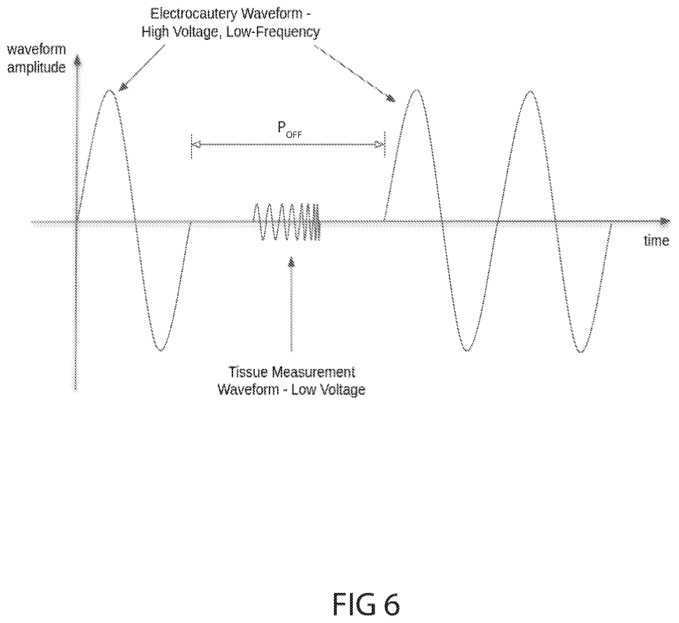

In some embodiments, probe 100 is used to both characterize tissue, and provide a treatment (e.g. deliver treatment energy 500), and console 200 utilizes time-interleaving and/or frequency-interleaving of the sensing signal and a treatment signal (e.g. an electrocautery signal or other treatment energy 500 signal) at a rate sufficient to produce the perception for the operator, that both are happening simultaneously and seamlessly (e.g. without additional effort), such as is represented in the interleaved drive signal 401 and treatment signal (e.g. delivery of treatment energy 500) of FIG. 6. A treatment energy 500 signal, such as an electrosurgical electrical signal at high-voltage and at frequencies in the range of 200 kHz to 3.3 MHz, can be interleaved with a tissue characterization drive signal 401, such as a low-energy signal at higher frequencies (e.g. up to 20 GHz). For example, the treatment energy 500 delivery can be duty-cycled, and it can include repeated “off periods” (e.g. period PoFF as shown in FIG. 6), where tissue characterization can be performed (e.g. the drive signal 401 is delivered and the recorded signals 402 are recorded) during these off periods. In some embodiments, console 200 includes isolation network 280 which safely and effectively switches between drive signal 401 delivery to electrodes 151, and treatment energy 500 delivery to electrodes 151. Isolation network 280 can be configured to deliver drive signals 401 to a first set of electrodes 151, and to deliver the treatment energy 500 to a second, different set of electrodes 151. The second set of electrodes 151 can comprise a larger, smaller, or equal quantity of electrodes 151, and/or coverage area of electrodes 151, such as to deliver the treatment energy 500 to a larger, smaller, or equal amount of tissue, respectively. Isolation network 280 can be configured to discharge components of system 10 after energy delivery (e.g. discharge any capacitance of cable 20, and/or any electrical current flowing in cable 20), such as a discharge of energy performed prior to delivery of a drive signal 401 to electrodes 151. The “off time” between two treatment energy 500 deliveries (e.g. in a duty cycle mode) can be relatively short, in the range of milliseconds, allowing drive signal 401 delivery during the off time, and providing the operator with continuous information on the tissue receiving treatment (e.g. receiving treatment energy 500). This alternating mode improves operator ease-of-use by eliminating the need to turn a tissue characterization mode on and off, by using an “always-on” characterization mode. This mode is compatible with both monopolar and multipolar treatment energy 500 delivery (e.g. electrosurgical or other treatment energy 500 delivery by probe electrodes 151 and/or treatment device 300).

Although system 10 is described with respect to delivery of drive signals 401 in the RF frequency range of from a few kHz to several GHz, signal generator 210 can (alternatively or additionally) deliver drive signals 401 of other frequencies and/or energy types, beyond the RF range, including microwave, ultrasound, and/or other electro-magnetic forms and frequencies of energy.

Console 200 includes controller 220, which includes various electronic and/or other components for interfacing (e.g. receiving signals from, and/or transferring energy and/or other signals to, probe 100 and/or other components of system 10). Controller 220 can include signal processor 230 which can be configured to perform one or more of: signal filtering (e.g. low-pass, high-pass, and/or band-pass filtering of signals); signal amplification; combining of signals; separating of signals; mathematical processing of signals; averaging of signals; identifying maximums and/or minimums in signals; transformation of signals, such as Fourier transformation of signals; integration and/or differentiation of signals; sampling of signals; copying of signals; compression of signals; convolution of signals; decomposition of signals; and/or other signal processing functions, such as signal processing functions performed on signals received from probe 100 (e.g. recorded signals 402 of the present inventive concepts) and/or treatment device 300. Controller 220 can further include one or more analog-to-digital converters, digitizer 240 shown, such as to transform analog-based recorded signals 402 to digital data.

Controller 220 can include memory 260 and/or algorithm 270, each as described herebelow.

In some embodiments, controller 220 comprises a mechanical linkage control assembly (e.g. functional element 299 configured as a mechanical linkage control assembly), such as to robotically or otherwise articulate tip 110, shaft 101, and/or another portion of probe 100, as described herein.

In some embodiments, controller 220 comprises a fluid control assembly (e.g. functional element 299 configured as a pneumatic and/or hydraulic control assembly), such as to robotically or otherwise control one or more pneumatic and/or hydraulic actuators of probe 100 and/or treatment device 300.

In some embodiments, controller 220 provides control signals to control treatment device 300 and/or a treatment portion of probe 100 (e.g. a functional element 199 configured as a treatment assembly). For example, controller 220 can provide control signals to adjust energy delivery (e.g. adjust the position of one or more portions of probe 100 and/or treatment device 300 delivering treatment energy 500), adjust drug or other agent delivery, adjust mechanical cutting parameters, adjust heating and/or cooling parameters (e.g. for cryotherapy tools), and/or adjust other treatment parameters of these devices.

User interface 250 is configured to provide information (e.g. TCI 400 and/or other information) to an operator of system 10 and/or to receive information from an operator of system 10 (e.g. surgeon or other clinician commands). User interface 250 can include one or more user input and/or output components. User interface 250 can include one or more user output components selected from the group consisting of: computer screen; tablet; phone; LCD; display such as a plasma display; touch screen; augmented and/or virtual reality displays; light; LED: an array of light emitters or LEDs; speaker; tactile transducer such as a vibrational transducer; and combinations of these. User interface 250 can include one or more user input components selected from the group consisting of: keyboard; keypad; mouse; joystick; microphone; switch; foot-activated switch; button; dial; camera; scanner; fingerprint scanner; retinal scanner; and combinations of these.

In some embodiments, system 10 is configured to provide audible feedback to an operator, such as an alert and/or other information delivered via a speaker or other audio output element of user interface 250. In these embodiments, multiple audible frequencies, signals, and/or audible words can be delivered, such as to indicate different tissues (e.g. to differentiate one tissue type from another), tissue characteristics, the presence or absence of tissues, and/or the probability of the presence of tissues and/or tissue characteristics. Alternately or additionally, pulsed audible signals can be provided, such as signals that vary in speed to indicate proximity to tissue (e.g. to identify a tissue surface or a particular tissue type), such as in the manner of the output of a backup sensor system for a vehicle.

In some embodiments, controller 220 comprises one or more memory storage components, memory 260. Memory 260 can comprise one or more forms of memory including but not limited to: read only memory (ROM); random access memory (RAM); volatile memory; non-volatile memory; memory included in integrated circuits; memory included on CD ROMs; and combinations of these. In some embodiments, signals recorded by probe 100 (e.g. recorded signals 402) and/or another device of system 10 are maintained (at least temporarily) in memory 260. In some embodiments, calculated information, such as TCI 400 is stored in memory 260. In some embodiments, information related to tissue type or other tissue characteristics (e.g. from the current patient or previous patients), is stored in memory 260.

In some embodiments, controller 220 comprises one or more algorithms, algorithm 270 shown. Algorithm 270 can be configured to provide (e.g. calculate) TCI 400 based on signals provided by probe 100, as described herein. Algorithm 270 can be configured to further process TCI 400, such as to produce a control signal to control treatment device 300 and/or a treatment portion of probe 100, also as described herein.

In some embodiments, one or more of functional elements 99, 199, 299, and/or 399 (of system 10, probe 100, console 200 and/or treatment device 300 respectively), comprises one or more sensors that provide sensor signals (e.g. signals related to a physiologic parameter of the patient, and/or signals related to probe 100, treatment device 300, and/or another component of system 10), and these signals are analyzed by algorithm 270 (e.g. after signal processing performed by signal processor 230), to generate one or more of: TCI 400; non-tissue information (e.g. patient or system information to be provided to an operator of system 10 via user interface 250); a control signal (e.g. an enable/disable, robotic, or other control signal used to control probe 100 and/or treatment device 300).

Controller 220 can include various “models” of tissue (e.g. as data stored in memory 260), these tissue models can be used (e.g. by algorithm 270) to differentiate tissue or groups of tissue. These tissue models can be based on previously gathered data (e.g. from the same patient and/or a previous one or more patients). The tissue models can comprise a “starting model” (e.g. a model which performs relatively well across a variety of different patients and conditions) that was previously “trained” (e.g. creating a “trained classifier”), such as by using reinforcement learning on a variety of labeled (known) tissues and/or chosen for use in system 10 based on validation data (e.g. labeled data not used in training) such as to prevent overfitting to the data used for training. The tissue models can be trained to classify one or more tissue types and/or tissue characteristics of interest, for example tissue that should be considered target tissue (e.g. tissue to be cut or otherwise treated) and/or non-target tissue (e.g. tissue to avoid cutting or otherwise avoid being treated). The tissue models can be selected and/or adjusted to represent tissues with a false negative bias, a false positive bias, a particular sensitivity, and/or a particular specificity. This selection and/or adjustment can be used for applications with different criticality of detecting the presence of one or more tissue types of interest.

Tissue models of controller 220 (stored in memory 260) can comprise an architecture that can be configured for use in a reinforcement learning model, such as a Neural Network, Support Vector Machine, Decision Tree Classifier, and/or Random Forest Classifier. A tissue model can include a Neural Network classifier that includes a minimum of two layers, such as an input layer and an output layer, and/or includes a minimum of three layers, and/or includes one hidden layer, as described herebelow. In some embodiments, system 10 can use a single tissue model to make a prediction (e.g. to produce TCI 400). In some embodiments, system 10 uses more than one tissue model of varying architecture to produce TCI 400, such as by using input from multiple tissue models' individual predictions. For example, system 10 can input the data from current tissue of interest into both a random forest model and a neural network, and then use the average of the probabilities for each tissue type between the two tissue models to then make a final prediction (e.g. to produce TCI 400). System 10 can primarily use one tissue model to make a prediction (for example, the tissue model with the highest validation score) and additionally use other tissue models to compare the predicted tissue probabilities output of the primary tissue model, for example as a safety check or other confirmatory step. In some embodiments, all or at least multiple of these outputs are provided to the operator of system 10.

In some embodiments, controller 220 comprises a neural network architecture comprising a “fully connected” network. In these embodiments, each “node” from one layer is connected to all other nodes of the next layer. This connection arrangement takes the form of a weight which is multiplied by the features (in the case of the input layer). The multiplication of the weight is a linear function. The neural network can then apply a non-linear function to the product of the features and weights called an “activation function”. This activation function can be a rectified linear unit (ReLU) which correlates to taking the maximum of the product and zero. The activation function can be a sigmoid function, or a hyperbolic tangent function (tanh), and/or some other non-linear function. By applying the linear function followed by a non-linearity, the neural network can approximate functions with increasing accuracy, as this pattern is repeated (more layers, or linear functions, followed by a non-linearity).

For example, at least two layers can be included in a neural network of controller 220, the network comprising an input layer and an output layer. The input layer comprises the feature set and has the same number of nodes as there are features. The output layer has the same dimensions as the output of the model, so for example if the model is designed to differentiate between X different tissues and/or tissue characteristics then the output layer will have X nodes. The output layer can apply a “soft max” function to the output which makes all the output values represent the probability of the input sample belonging to each tissue and/or tissue characteristic class. A two-layer neural network will be able to effectively approximate relatively simple functions.

In another example, at least three layers can be included in a neural network of controller 220. A three-layer network includes the inner and outer layer of the two-layer network described immediately hereabove, as well as a “hidden” layer between the input and output layers. The hidden layer can have at least one node and can have more nodes than the output layer. The hidden layer is fully connected to each layer before and after it, and similarly has a weight for each connection followed by the application of a non-linear function. A three-layer network enables more complex function to be practically approximated. Additional layers can be included in the neural network of controller 220 to further enable improved or more complex function approximation.

The weights from a trained neural network of controller 220 could be stored on and executed from memory 260 (e.g. memory 260 comprising flash, EEPROM, hard drive, and/or RAM). During a clinical procedure, the weights, activation function(s), and architecture can be used to perform the necessary calculations using one or more components of controller 220 (e.g. MCU, MPU, CPU, GPU, ASIC, FPGA, discrete logic, and/or other processor/circuitry capable of performing the required calculations).

Before use in a surgery and/or other clinical procedure, system 10 can benefit from one or more measurements from the patient (e.g. one or more sets of recorded signals 402 of the present inventive concepts) to be used in the following ways: use of samples to verify the existing tissue model(s) are correctly predicting the samples; retraining of tissue models to fit the (current) patient; alteration of the input of future samples before they are input to a predicting tissue model; and/or use of samples to alter previously collected samples and then retrain the tissue model based on the new altered samples.

Once stored in memory 260, a tissue model can use recorded signals 402 from probe 100 to make real-time predictions. For example, console 200 (using a tissue model or otherwise) can be configured to produce TCI 400 within a short period from the recording of recorded signals 402, such as within a time period of no more than 5 seconds, no more than 2 seconds, and/or no more than 50 milliseconds (e.g. including producing TCI 400 representing tissue in contact with probe electrodes 151 and/or offset but proximate the contacting tissue). System 10 can include one or more tissue models which use the particular probe 100 type used as an input feature (e.g. when system includes multiple probes 100 of different types). One tissue model can be used to produce TCI 400 for multiple probes 100 (e.g. all probes 100), and/or multiple tissue models can be included to accommodate different sets of one or more different probe 100 types. In some embodiments, console 200 is configured to automatically detect a particular type of probe 100, and then associate an appropriate tissue model or models for use with that probe 100. A tissue model can use some or all of the following features which relate to the signal response from the tissue: electrical response; derivative of tissue response (1st, 2nd, or 3rd order); integral of tissue response; temperature of the tissue; type of probe 100; patient electrocardiogram information; optical properties of the tissue such as color or texture; force or pressure being applied at the tip 110; other patient information (e.g. gender, age, race, ethnicity, body mass index (BMI), body composition, one or more medical conditions, hydration level, temperature, electrolyte concentrations weight, and/or other biological characteristics); and/or environmental parameters (e.g. temperature, humidity, pressure, environmental electromagnetic radiation, elevation, location, and the like).

System 10 can determine the probabilities or likely types of the sample tissue belonging to a certain class, or it can indicate the most likely tissue(s) that are present in neighboring tissue. For tissue with an unacceptable outcome associated with “false negative” occurrences when the tissue is treated, the tissue can be identified as non-target tissue (e.g. tissue which is not desired for treatment or other effects of treatment). For example, system 10 can be configured so that the threshold required for that particular tissue type to be indicated is lowered to where the “True Positive Rate” from training and validation is near 100%, with very few false negative occurrences, at the cost of a higher “false positive” rate. The one or more tissue models can be configured to detect any fault in the probe 100 continuity separate from any other fault detection in the rest of system 10.

System 10 can be configured to disable a treatment (e.g. cutting, coagulation, heating, cooling, compressing, and/or other delivery of treatment energy 500) if certain tissue is detected, including devices including individual cutting instruments arranged in an array across the tissue.

System 10 can include multiple probes 100 where system 10 generates TCI 400 including a tissue type prediction for the neighboring tissue.

System 10 can be configured such that an operator can input corrected tissue characterization information, such as in the case of TCI 400 including incorrect or less certain tissue type predictions. This information could be used to update the tissue model for future predictions of TCI 400, including TCI 400 including future predictions provided during the same clinical procedure.

In some embodiments, system 10 creates individualized tissue models, and uses these models with a training set of data that more closely matches a specific patient or the demographics of a specific hospital, area, patient population, and the like.

System 10 can include one or more functional elements, such as functional element 99, functional element 199 (of probe 100), functional element 299 (of console 200), and/or functional element 399 (of treatment device 300). Functional elements 99, 199, 299, and/or 399 can comprise one or more sensors, one or more transducers, and/or one or more other functional elements.

In some embodiments, functional element 99, 199, 299, and/or 399 comprises a wireless element configured to transmit and/or receive information wirelessly (e.g. a Bluetooth or other wireless communication element that transfers information and/or power between one or more components of system 10).

In some embodiments, functional element 99, 199, 299, and/or 399 comprise an actuator, such as an electrical, mechanical, and/or fluidic (e.g. hydraulic or pneumatic) actuator, such as an actuator configured to robotically or otherwise articulate one or more portions of probe 100 and/or treatment device 300, as described herein.

In some embodiments, functional element 99, 199, and/or 399 comprise an element configured to perform a medical treatment (e.g. a treatment delivered to tissue), such as a treatment element selected from the group consisting of: a surgical or other treatment instrument; a needle or other access device; an energy delivery element; a monopolar energy delivery element; a bipolar energy delivery element; a drug or other agent delivery element; a fluid delivery element (e.g. a needle, a fluid jet, a porous balloon, and/or an iontophoretic fluid delivery element); a tissue cutting element; a tissue removal element; a heating or cooling element; an element configured to denature tissue; a grasper; a blade; a radiation delivery element; a fluid ablation element; and combinations of these.

In some embodiments, functional element 99, 199, and/or 399 comprises a hydrogel, such as is described herebelow.

In some embodiments, functional element 199, 399, and/or 99 comprises a memory storage element, such as a memory storage element used to store configuration information regarding probe 100, treatment device 300, and/or another component of system 10, respectively. The configuration information can be received by console 200, such as to configure console 200 for particular use (e.g. customized used) with the associated component.

In some embodiments, functional element 199 comprises a component selected from the group consisting of: a coupling feature for a calibration tool (e.g. tool 40 comprising a calibration tool); an analog to digital converter; an accelerometer; a magnetometer; a gyroscope; one or more fiducials for visual or magnetic location determination; a wireless communications element (e.g. a transceiver used to allow probe 100 to work without a connecting cable 20); a power source such as a battery and/or capacitor (e.g. a power source that provides a drive signal 401 and/or treatment energy 500 to electrodes 151 and/or other components of probe 100); and combinations of these.

In some embodiments, functional element 99, 199, and/or 399 comprise one or more “functional assemblies” as defined hereabove.

In some embodiments, system 10 further comprises one or more treatment devices, treatment device 300. Probe 100 can comprise all or a portion of treatment device 300 (e.g. probe 100 is configured as a multi-purpose device). Treatment device 300 can be configured to deliver one or more different forms of energy, treatment energy 500. Treatment energy 500 can comprise an energy type selected from the groups consisting of: electromagnetic energy; electrical energy; magnetic energy; sound energy (e.g. subsonic energy and/or ultrasound energy); light energy (e.g. laser light energy); mechanical energy (e.g. cutting energy and/or vibrational energy); chemical energy; and combinations of these. Treatment device 300 (e.g. probe 100) can comprise one or more treatment devices selected from the group consisting of: a cutting instrument; a cauterizing instrument; an electrocautery instrument; an ablation instrument; an electroablation instrument; a cryoablation instrument; a heating or cooling instruments; a light delivery instrument; a laser light delivery instrument; a harmonic scalpel; a vibratory instrument; a dissection instrument; a blunt dissection instrument; a scalpel-based instrument; a needle-based instrument; an aspiration instrument; a drug and/or other agent delivery instrument; a radiation delivery instrument; an instrument comprising a fluid jet; a fluid ablation instrument; and combinations thereof.

Alternatively or additionally, treatment device 300 can be configured to perform a diagnostic procedure on a patient, such as the patient from which system 10 produces TCI 400 and/or a different patient. For example, treatment device 300 (e.g. a treatment device 300 incorporated into probe 100) can be configured to monitor nerve evoked potentials, such as somatosensory nerve evoked potentials. Treatment device 300 can be a diagnostic device configured to monitor one or more physiologic parameters selected from the group consisting of: a blood parameter; a blood gas parameter; blood glucose; pH; respiration; heart electrical activity; blood pressure; temperature; and combinations of these.

Treatment device 300 can comprise one or more robotically controlled treatment devices, such as robotic control that is performed based on TCI 400 and/or other information recorded and/or produced by system 10.

In embodiments in which treatment device 300 and probe 100 comprise separate devices, and during use, delivery of treatment energy 500 by treatment device 300 can be performed at the same or at least proximate a location to which drive signals 401 are delivered (e.g. such that the tissue considered for treatment by treatment device 500 comprises the tissue represented by TCI 400).

TCI 400 can represent tissue types and other tissue characteristics from various forms of tissue in one or more locations of a patient.

The tissue characterization systems of the present inventive concepts, system 10, have application in reducing clinical procedure time and/or preventing surgical or other complications in a wide variety of clinical procedures. The following examples are presented as illustrative of the types of relevant clinical procedures and tissue types and/or other tissue characteristics that can benefit from the use of system 10.

In various clinical procedures, certain tissue is to be treated (“target tissue”), and other tissue is to avoid being treated or otherwise adverse effected by treatment (“non-target tissue”). In some procedures, muscle and/or fat are target tissues (e.g. tissues that are acceptable to be cut during the procedure), and tissues such as nerves, arteries, glands and/or ducts are non-target tissue. These non-target tissue can be hidden beneath target tissue, and/or these tissues can be difficult to visually identify (e.g. be difficult to avoid being cut). Therefore, in today's procedures, the surgeon often needs to move very slowly through tissue, carefully trying to dissect or cut target tissue, while attempting to identify non-target tissue (to avoid cutting). Via the tissue characterization information provided by system 10, TCI 400, the surgery (or other clinical procedure) can proceed more quickly and safely, such as when system 10 is set to provide a warning (e.g. via user interface 250) as the operator approaches or contacts non-target tissue (e.g. any tissue type that is not desired to be treated).

Nerve damage can cause a patient significant pain and other complications. In some locations of the body where there is a high concentration of nerves, such as the head and neck, surgical procedures can be very slow due to the need of the surgeon to perform delicate dissections to locate and subsequently avoid nerves. For example, system 10 can be configured to perform a clinical procedure on a patient's thyroid, such as to avoid damaging laryngeal nerves (a relatively common adverse event in today's thyroid procedures). Nerve monitoring devices are sometimes used to look for nerves in thyroid or facial surgery. However, nerve monitoring devices require that a sensing portion of the monitor be placed ahead of time and at a tissue innervated by the nerve that would respond to a stimulating portion from the monitoring device. Therefore, the TCI 400 provided by system 10 would be extremely useful, such as by providing identification of any nerve tissue (e.g. without the downstream innervated tissue access required by a monitoring device). Other applicable areas include the feet, hands, spine, and prostate of the patient, where similar advantages of using system 10 for nerve identification and/or detection can be realized.

Gland tissue can be difficult to locate because it may appear similar to, and thus may be mistaken for, fat and/or other non-glandular tissues. Identification of gland tissue via system 10 can be useful in preventing or at least reducing damage to gland tissue, which subsequently correlates to reduced overall complications in procedures performed near gland tissue.

Lymphatic tissue, including lymphatic ducts and nodes, can also be difficult to visually identify. Lymphatic ducts are present throughout the body, and these tissues can be identified by system 10 to provide the advantages described herein.

Arteries and veins, when accidentally cut or damaged during surgery, can result in complications and extend surgical time as the surgeon must take the time to control and eventually stop the bleeding, as well as repair the associated vessels. Locating these vessels using system 10 allow the operator to both work more quickly and avoid complications.

Identification of fascia, tendons, and/or bones by system 10 could also be useful to an operator performing a surgery or other clinical procedure, such as when these tissue types are target tissue to be treated, and/or non-target tissue to be avoided.

Calcification occurs in arteries, tendons, and other structures, and is sometimes removed during a surgical or other clinical procedure. Identification of calcification by system 10 (e.g. TCI 400 includes characterization of a tissue as being calcified), can optimize the associated clinical procedures (e.g. through improved efficiency and/or reduced complications).

Removal of tumors or cancerous tissues surgically involves differentiating between healthy tissue (e.g. non-target tissue) and cancerous tissue (e.g. target tissue), and the differentiation is often very time consuming and sometimes of limited effectiveness. Determination of the of tumor versus normal, healthy tissues, as well as the margins of tumor tissue, all using system 10, will allow an operator to improve both accuracy of treatment (e.g. correlating to improved outcomes), and speed of surgery.

During electrocautery procedures, it is sometimes important to avoid burning skin tissue. In order to suture skin with minimal scaring and optimized healing, skin should be cut rather than cauterized. System 10 can be configured to prevent an operator from delivering energy to the patient's skin (e.g. via treatment device 300 and/or a probe 100 configured to deliver electrocautery energy), resulting in improved outcomes and reduced procedure time.

Ducts, for example the ureter, pancreatic duct, and/or bile duct, transmit fluid from a gland or organ. Accidental cutting of a duct can lead to serious complications when this fluid subsequently passes into other, non-desired parts of the patient's body. Since these ducts can sometimes be difficult to distinguish (e.g. visually distinguish) from other tissues within the surgical site, use of system 10 to identify and avoid duct tissue can reduce complications and reduce procedure time.

Some further types of tissue that can be identified or otherwise characterized by system 10 include: scar tissue; tissue that has already been treated; dead tissue; and/or living tissue (e.g. TCI 400 includes information to differentiate dead versus living tissue). Additionally, system 10 can be configured to provide tissue temperature information. The temperature of the tissue can be identified (e.g. continuously and/or in real time), and this temperature information can be used in providing further tissue identification information. In some embodiments, one or more probe electrodes 151 and/or functional elements 199 and/or 299 comprise a thermocouple or other temperature sensor configured to provide tissue temperature, bodily fluid temperature, and/or other temperature information (e.g. patient temperature or system 10 components temperature information). In some embodiments, probe 100 and/or treatment device 300 includes an infrared sensor configured to provide tissue or other temperature information to console 200 (e.g. temperature information to be used by algorithm 270 and/or signal processor 230).

System 10 can be operated or otherwise used by one or more surgeons, clinicians, and/or other operators (“operator” or “operators” herein).

In some embodiments, probe electrodes 151 comprise a set of electrodes configured for bipolar or other multipolar energy delivery (e.g. as provided by signal generator 210). In these embodiments, signal generator 210 can be also configured to deliver monopolar-based treatment energy 500 to one or more probe electrodes 151 (e.g. when system 10 further includes electrode 30 configured as a return electrode). In these embodiments, probe 100 can be further configured as an electrocautery device, where probe electrodes 151 and/or other included electrodes deliver monopolar and/or bipolar energy to perform electrocautery functions such as tissue cutting, tissue ablation, and/or hemostasis (e.g. via coagulation of blood with heat). The monopolar and/or bipolar electrocautery energy can be provided by signal generator 210 (e.g. in addition to providing the signals used to characterize tissue) and/or another energy providing component of system 10. Console 200 can include an electrically switchable isolation network, isolation network 280, which can be used to independently connect probe electrodes 151 to one or more portions of signal generator 210 and/or other portions of console 200 (e.g. to safely alternate connections between a first source of energy (e.g. a source of drive signals 401) and a second source of energy (e.g. a source of electrocautery or other treatment energy 500)), while providing isolation from each other when not connected. For example, the isolation network 280 can electrically separate probe electrodes 151 during tissue characterization (e.g. into one or a few number of probe electrodes 151), and then connect them together during electrocautery (e.g. to a larger number of probe electrodes 151 to maximize electrode area during electrocautery treatment).

The challenge of transmitting both tissue characterization signals and a treatment energy 500 (e.g. electrocautery energy) over a thin and flexible cable can be addressed by console 200 multiplexing both of these signals through a single impedance-controlled electrical connection. Isolation network 280 can include active and/or passive switch networks that are configured to achieve multiplexing of treatment and measurement signals while protecting the patient, the instrument, and/or the operator from high-voltage waveforms. In some embodiments, isolation network 280 is configured to provide mitigation of harmonic content from a nonlinear network, such as when isolation network 280 includes a filter network that consists of passive electrical filter elements.

In some embodiments, probe 100 includes a reusable handle 101 and/or shaft 102, and a disposable tip 110. The disposable tip 110 is removable from the remaining portion of probe 110, and can be replaced for each clinical procedure in which system 10 is used. System 10 can include multiple tips 110, each with a different configuration, such as a different length, shape and/or a different arrangement of probe electrodes 151 (e.g. different configurations for use to characterize specific tissue types and/or to be used in particular portions of the patient's anatomy). The tips 110 can be connected to the remaining portion of probe 100 by frictional engagement (e.g. a snap fit), mating threads, and the like.

In some embodiments, tip 110 is configured for use in multiple clinical procedures (e.g. performed on multiple patients), but is further configured to be removed, such as to perform a cleaning procedure after each use. The entire probe 100 can be disposable and/or reusable. In some embodiments, a portion or all of cable 20 is disposable. In some embodiments, a tip 110 configured to be disposable includes a functional element 199 comprising a data storage element, such as for storing information pertaining to the particular operation of that tip 110, such as information received by console 200 to configure console 200 (e.g. to configure signal generator 210) for use with the tip 110. For example, specific information related to probe electrodes 151 of tip 110 can be used by console 200 for its configuration (e.g. configuration of drive signals 401 and/or recorded signals 402).

As described hereabove, all or a portion of console 200 can be included in probe 100 (e.g. contained within a housing a probe 100). In some embodiments, system 10 does not include cable 20, and probe 100 communicates with one or more portions of console 200 via wireless transmission. For example, probe 100 can include a functional element 199 comprising a first functional element 199a comprising a battery and/or other energy storage component and a second functional element 199b comprising a wireless transmitter and/or receiver. Functional element 199a can provide energy for delivering drive signals 401, for delivering treatment energy 500, and/or for providing electrical energy to one or more electronic components of probe 100. Functional element 199b can be configured to transfer information between probe 100 and console 200 and/or another component of system 10.

System 10 can be calibrated before use on a patient, such as to adjust for changes in the signal between the probe electrodes 151 at tip 110, and the signal received by the console 200 (e.g. recorded signals 402 herein). Cable 20 and probe 100 can both be sources of changes in the characteristics of the signal between the probe and the controller 220. To ensure that the recorded signals 402 used in tissue identification do not include distortion and/or console 200 accounts for variance caused by the recorded signals 402 traveling along probe 100 or cable 20, a calibration step can be performed. Alternatively or additionally, in some embodiments system 10 includes materials and/or wire winding techniques configured to reduce distortion and/or other adverse signal effects. In some embodiments, cable 20, probe 100, and/or other components of system 10 include twisted pair wiring and/or micro coax cabling, such as noise canceling cabling configured to reduce the effects of cable orientation, cable movement, and/or electromagnetic interference.

In some embodiments, calibration is performed using a bilinear transform to calibrate probe 100 based on the use of a known resistor, tool 40 (e.g. a known calibration load comprising one or more resistive elements). Tool 40 is placed between probe electrodes 151 during calibration, such as to represent a known tissue impedance. Calibration is performed by adjusting one or more components of console 200 to create a desired output based on the known impedance.

The bilinear transform can be used to perform a mathematical mapping of electrical measurements made by console 200 to values of the recorded signals 402 made by probe electrodes 151. For example, analog-to-digital converters (ADCs) can be included in console 200 to convert analog-based recorded signals 402 into digital signals. These recorded and converted signals are useful but can contain parasitic values associated with cabling 20 and probe 100. The bilinear transform can be configured to remove source of noise and other parasitic signal components, and create data representing the true signals (e.g. actual voltage and current) present at probe electrodes 151. In order to perform this task, a tool 40 comprising a set of known calibration loads can be placed in electrical connection with probe electrodes 151, with measurements made at console 200. This process, “calibrating the transform”, once performed, produces a set of constants that are applied to the transform (a mathematical transform used to produce TCI 400 based on drive signals 401 and recorded signals 402). This calibrated transform can be used to improve signal integrity, such as to improve the accuracy of recorded signals 402, such as to improve the accuracy of TCI 400 related to tissue in contact with and/or otherwise proximate probe electrodes 151 (e.g. the neighboring tissue).

System 10 can be configured to perform a calibration that compensates for PtoP differences and/or temporal differences (within a single patient) that relate to tissue characteristics to be assessed (e.g. TCI 400 to be produced). For example, PtoP differences can be present in two or more volumes of the same tissue type, such as due to varied hydration status of the (single) patient. P2P differences can correlate to differences in a patient parameter selected from the group consisting of: age; ethnicity; tissues composition; temperature; electrolyte concentration; disease state; medical history; medications taken; diet; patient demographics; salinity; and combinations of these. The step of setting the patient-specific tissue reference can involve contacting probe electrodes 151 of probe 100 with one or more known types of patient-specific tissue and/or bodily fluid prior to use of system 10 to characterize tissue. The step of setting the patient-specific tissue reference can be used to adjust system 10 for patient-specific differences in tissue characteristics (e.g. tissue impedance or other characteristic for the same tissue type) and adjust the output of system 10 based on the tissue reference. This step can greatly improve the accuracy and precision of the tissue type characterization (e.g. the precision of TCI 400). In one example, system 10 is calibrated by placing the probe electrodes 151 of probe 100 in contact with and/or in known proximity to (“in contact with” herein) a first known tissue type (e.g. muscle tissue), recording the results, and then placing probe electrodes 151 in contact with a second type of tissue (e.g. fat tissue), and recording the results. These recordings can then be used as a baseline reference value (e.g. stored in memory 260 and/or used by algorithm 270) for the particular patient and the tissue characterization for that particular patient can be adjusted based on the results of this calibration. In this way, if system 10 is being used for sensing proximity to nerves during a clinical procedure, the operator does not have to specifically locate a nerve and calibrate the system with nerve tissue. Alternatively, system 10 can be calibrated using one or more known tissues that are more easily located and identified. For example, the operator can contact probe electrodes 151 to an identified nerve and/or gland to calibrate system 10 to efficiently identify other nerves and/or glands, respectively. In some embodiments, calibration is performed on a single known tissue type. In other embodiments, calibration is performed on two, three, or more known tissue types.

In some embodiments, a patient tissue baseline reference is determined using system 10 using a bodily fluid, such as blood drawn from the patient. The tissue baseline set with bodily fluid extracted from the particular patient (e.g. a patient to which a clinical procedure is to be performed in which system 10 is used to characterize tissue of that patient) can be done preoperatively, such as within a relatively short time frame of the clinical procedure (e.g. within one hour, within 12 hours, and/or within 24 hours), since tissue characteristics or response can be highly sensitive to patient hydration or other relevant patient parameters which can change over time. Alternatively or additionally, a tissue baseline can be determined (e.g. using muscle, fat, and/or other internal tissues) can be performed during the clinical procedure. In some embodiments, console 200 is configured to request the operator obtain tissue baseline information (e.g. by placing probe electrodes 151 on known tissue) on a routine basis, such as within a particular time duration, in a repeated manner. In these embodiments, console 200 can be configured to require the operator to obtain this information (e.g. in the repeated manner), such as when treatment (e.g. delivery of treatment energy 500 and/or other treatment performed by probe 100 and/or treatment device 300) is prevented until the baseline information is properly gathered.

A patient tissue baseline reference performed using system 10 can also involve inputting to the console 200 (e.g. via user interface 250) some patient-specific data such as data related to a patient parameter selected from the group consisting of: age; height; weight; ethnicity; electrolyte concentration; blood data (e.g. data resulting from one or more blood tests); and combinations thereof. This patient-specific data can be used alone or in combination with tissue contact reference information. Patient tissue baseline reference information can be stored in memory 260 of console 200, such as in a database used for predictive identification of a correspondence between patient-specific data, feedback, and specific tissue characteristics used for producing TCI 400. In some embodiments, console 200 is configured such that one or more functions (e.g. delivery of energy 500 and/or other treatment using probe 100 and/or treatment device 300) is prevented until the above patient-specific data is entered into console 200 by the operator.

The patient tissue reference step and corresponding calibration of system 10 can include initial calibration only or continued or periodic calibration by system 10 that occurs during the clinical procedure (e.g. repeatedly on an operator-determined and/or system-determined time interval). For example, console 200 can be configured to use machine learning (e.g. via algorithm 270) which can be combined with TCI 400 to allow continued learning and calibration of system 10 based on sensed data (e.g. in addition to TCI 400) and/or operator tagged or otherwise identified tissues throughout the clinical procedure. The operator can identify tissue during the procedure, which can result in improved accuracy of system 10 (e.g. system 10 more accurately characterizing tissue).

In some embodiments, one or more different tissue types are associated with a predetermined range of each of a plurality of classification parameters. These classification parameters for various tissue types can include one or more measured and/or calculated parameters, including voltage, waveform, power, frequency, phase, duty cycle, duration, energy, impedance, inductance, and/or capacitance. The ranges of expected values for these classification parameters for each can form a type of generic multidimensional (e.g. 2D, 3D and/or or higher dimensional) map of the different types of tissue for a generic patient and/or set of patients. This “generic tissue map” can be determined from patient data collected by system 10, such as is described herein, and/or other patient collected data. A patient-specific baseline reference can be used by system 10 (e.g. by algorithm 270) to adjust the generic map of tissue types for the particular patient (e.g. at a particular timepoint). The generic map can be adjusted for the specific patient in a number of ways, including reducing variation in the generic identified range for each parameter to a more specific patient map or series of ranges (“map” herein) for each type of tissue. Alternately, patient-specific baseline reference information can be used by system 10 to perform a shift of the map to values more specific to the patient (e.g. and the particular timepoint).

Console 200 can be configured to create a spatial map of identified tissues and other tissue characterization data, using TCI 400 and localization information related to probe electrodes 151 (e.g. multi-dimensional localization information related to probe electrodes 151). This spatial map can be localized in reference to other known structures (e.g. other known tissue and/or other structures of the patient). Multidimensional localization of probe electrodes 151 can be accomplished using imaging, computer vision, magnetic navigation, and other similar navigation techniques (e.g. when tool 40 comprises an imaging device, (e.g. as described hereabove). The localization can be accomplished with, or without, the use of markers (e.g. functional elements 199 and/or 299 comprising markers), such as fiducial markers, magnetic, visual or retroreflective markers.

In some embodiments, probe 100 is configured to both record data to characterize tissue (e.g. to produce TCI 400), as well as perform another medical procedure, such as an additional patient diagnostic procedure and/or therapeutic procedure (e.g. probe 100 includes all or a portion of treatment device 300). For example, probe 100 can be further configured as a device selected from the group consisting of: a cutting instrument; a cauterizing instrument; an electrocautery instrument; an ablation instrument; an electroablation instrument; a cryoablation instrument; a heating or cooling instrument; a light delivery instrument; a laser light delivery instrument; a harmonic scalpel; a vibratory instrument; a dissection instrument; a blunt dissection instrument; a scalpel-based instrument; a needle-based instrument; a drug-delivery instrument; an aspiration instrument; and combinations of these. Probe 100 can be configured to cause effective treatment of target tissue (e.g. effective delivery of treatment energy 500 to target tissue) and/or to prevent undesired treatment of target tissue (e.g. inadequate, misdirected, and/or other undesired delivery of treatment energy 500 to target tissue). Probe 100 can be configured to cause treatment of target tissue only, while protecting non-target tissue from undesired effects (e.g. prevention of undesired delivery of treatment energy 500 to non-target tissue).

When probe 100 is further configured as an electrical instrument (e.g. an instrument configured to deliver an electrical signal or other electrical energy, such as an electrocautery, electrosurgery, and/or other electrical energy delivery instrument), functional element 199 can comprise one or more electrodes used to deliver electrical energy to the patient. Alternatively or additionally, one or more of probe electrodes 151 used for gathering TCI 400 can be the same as the electrode(s) used for delivering electrical energy, treatment energy 500, to the patient (e.g. used for tissue treatment). A probe 100 configured for both tissue characterization and performing a treatment procedure (e.g. a treatment in which treatment energy 500 is delivered to tissue) can be configured for open surgical procedures, for robotic procedures, and/or for laparoscopic or other minimally invasive procedures. Utilizing the same probe electrodes 151 for each function, and/or electrodes (e.g. functional element 199) in close proximity to the probe electrodes 151 for the treatment function, is beneficial in that there is a higher correlation between the tissue being characterized and the tissue being treated and/or diagnosed (e.g. due to the close proximity).

In some embodiments, probe 100 is configured to gather recorded signals 402 that is used by system 10 to determine when an included needle and/or other access device (e.g. functional element 199 comprises a needle and/or other access device) is positioned in desired tissue to be treated and/or at a particular desired treatment location within the desired tissue. This configuration can be used for determining if the access device(s) is in a desired tissue location, such as for taking biopsies, placing a vascular (e.g. arterial) line, performing a lumbar puncture, locating nerves (e.g. for injection of medication or other material), for intralumenal injections, for extraction of material, and/or for determining access device depth within tissue and/or distance from fluid bodies. In some embodiments, one or more electrodes are positioned on the needle and/or other access device, such as to gather tissue characterization information related to the tissue in contact with the access device at the time.

In some embodiments, a probe 100 that includes a needle and/or other access device (e.g. a functional element 199 comprises an access device) can be configured to provide feedback (e.g. via user interface 250) about a location of the access device within tissue, and/or a warning when the access device has exited the tissue. For example, the needle or other access device portion of probe 100 can include electrodes 151 (e.g. tissue-contacting electrodes) for delivering drive signal 401 and/or recording the recorded signal 402. Controller 220 can analyze recorded signals 402 (e.g. changes in recorded signals 402) such as when the access device nears an edge (e.g. an edge of a layer of heart or other tissue). This feedback can be used for properly locating a needle in the heart wall (e.g. TCI 400 indicates heart wall tissue), such as for injections of one or more materials into the heart wall. For example, functional element 199 can further comprise one or more hydrogels that can be injected by probe 100 into a heart wall (e.g. arthroscopically after heart attack) to provide mechanical support to stabilize the damaged area. The hydrogel can be configured to limit formation of scar tissue, limit thinning of the heart's walls, and/or limit enlargement of the heart. To determine a distance that a needle or other access device of probe 100 has advanced through tissue, such as a heart wall, console 200 can measures a change in TCI 400 as the access device is advanced into a layer of tissue, and console 200, via user interface 250, can provide information indicating when the access device is nearing an edge of the layer of tissue and/or getting close to a fluid body.

When probe 100 further comprises a surgical instrument or other treatment instrument (e.g. probe 100 comprises all or a portion of treatment device 300 and/or functional element 199 comprises the treatment instrument), probe electrodes 151 can be positioned just proximal the distal end of the instrument (e.g. just proximal the distal end of a functional element 199 configured as the instrument) or otherwise positioned on probe 100 such that probe electrodes 151 are in close proximity to the location of treatment (e.g. such that when the instrument-portion of probe 100 is drawn through tissue in manner of a knife or other cutting instrument, the tissue can be identified just prior to cutting or otherwise treating).

In some embodiments, one or more probe electrodes 151 are used to both cut and cauterize tissue, as well as gather TCI 400. In these embodiments, console 200 can comprise a foot-activated switch and/or other switch (e.g. user interface 250 include comprises a foot-activated switch and/or other switch) that allows an operator of system 10 to easily switch between a tissue identification mode and a tissue treatment mode. The treatment mode can use a higher power and/or longer duration of energy delivery from probe electrodes 151 than the identification mode. In some embodiments, console 200 comprises isolation network 280 to assist in the safe and effective transition between delivery of drive signals 401 for tissue characterization and delivery of treatment energy 500 (e.g. each by probe electrodes 151).

In some embodiments, probe 100 can be configured to gather TCI 400 (e.g. via recordings made by probe electrodes 151) as well as to perform electrostimulation of nerves (e.g. via energy delivery via probe electrodes 151 and/or functional element 199 configured to deliver electrical or other stimulation energy). In this configuration, tissue classification can be performed via delivery of signals with a frequency above 100 kHz, and the electrostimulation of nerves can be performed (e.g. with the same probe electrodes 151 or otherwise) with a frequency set below 100 kHz.

In some embodiments, system 10 is configured to provide an “intelligent action function” (e.g. probe 100 and/or treatment device 300) is configured as an “intelligent action instrument”), where system 10 uses TCI 400 to affect (e.g. in a feedback mode performed manually by the operator, or automatically by system 10) the action being performed on the tissue. For example, based on the feedback provided, the system 10 and/or the operator may avoid treating (e.g. avoid cutting) specific tissue and/or only treat a limited amount of tissue (e.g. treat specific tissue). System 10 can be configured such that the therapeutic function (e.g. energy delivery) is automatically enabled and/or disabled, based on TCI 400 (e.g. produced and/or analyzed by algorithm 270). This intelligent action or automatic enable/disable feature can further include system 10 providing other tissue characterization feedback, in addition to the enable/disable feature. The intelligent action function can be used to prevent cutting, cauterizing, and/or otherwise modifying specific tissue during a clinical procedure. The tissue to avoid modifying can include one or more of nerve, artery, vein, lymphatic tissue, gland, tendon, skin, duct, fat, and the like. Alternatively or additionally, the intelligent action function can be configured to cut or otherwise treat only particular types of tissues, such as only treating muscle, fat, skin, and/or bone. The intelligent action function can also be configured to “tune” a tissue treatment, (e.g. tune delivery of treatment energy 500 for cutting, cauterization, ligation, and the like), such as by adjusting to levels of energy-delivery and/or other parameters that are better suited to a particular type of tissue. Examples of relevant parameters to be adjusted include waveform, power, frequency, voltage, phase, duty cycle, depth of treatment, and/or temperature. A probe 100 configured as an intelligent action instrument can safely and efficiently allow an operator to dissect around one or more certain tissue types, such as nerves, bone, vessels, tumors, cancerous tissue, glands, fascia, and/or lymphatic tissue.

In some embodiments, probe 100 can be configured as an intelligent action instrument comprising a bipolar surgical device, such that probe 100 is further configured to record TCI 400 (e.g. this information is used by system 10′s intelligent action function as described hereabove). Probe 100 can be configured as a bipolar cutting instrument, such as when functional element 199 comprises scissor-like electrical elements including probe electrodes 151, which can be used to grasp tissue and cut the tissue between the scissor-like elements (e.g. via bipolar energy delivered between the two elements). Capturing unwanted tissue, such as blood vessels, ducts, and/or nerves with a bipolar cutting device can result in unwanted complications. Since these structures can be hidden beneath other tissue and/or are otherwise difficult to identify, a probe 100 configured as an intelligent action device can provide significant advantages. Such a probe 100 configured as an intelligent action bipolar cutter can detect these tissues within tissue trapped between the electrodes and either warn the operator (e.g. via an alert provided on user interface 250) and/or automatically disable the energy delivery function of probe 100 to prevent undesired cutting of the tissue. System 10 can be configured to identify tissues of interest even if they lie underneath the tissues directly on the visible surface, as described herein.

In some embodiments, probe 100 is configured as an intelligent action instrument comprising a non-electrical treatment tool, such as a non-electrical treatment tool selected from the group consisting of: cutting tool; water jet blade; auto-retractable blade (e.g. a blade is automatically retracts based on TCI 400 produced related to tissue proximate the blade); ligating tool; stapling tool; needle based tool; injection tool; heat ablation tool; cryoablation tool; pressure-delivery tool; balloon-based tool; and combinations of these. In these embodiments, TCI 400 can be used to automatically enable, disable, and/or otherwise modify the operation of the non-electrical treatment tool.

System 10 can be configured to trigger an output signal to the operator under certain conditions, the output signal provided by user interface 250 and including: an audible signal; a visible signal such as a displayed icon or a light (e.g. a flashing light); a sensory notification, such as a vibration; a text or numerical value related to tissue in proximity to probe electrodes 151 (e.g. a value related to the distance to the tissue; a value representative of the tissue type; and/or a value related to the tissue characteristics). User interface 250 can display numerical values, text, and/or graphical representations (e.g. icons, charts, and/or figures), which can be interpreted by the operator of system 10. The output provided by user interface 250 can specifically identify types of tissue and/or provide other TCI 400. Alternatively or additionally, user interface 250 can provide information related to a change in tissue type, a change in a tissue characteristic, and/or another change, such as to alert the operator that one or more tissue characteristics are changing (e.g. changing as the tip 110 location moves in tissue and/or along a tissue surface). System 10 can be configured to provide an alert (e.g. a warning or other notification) as particular types of tissues are identified. For example, a positive feedback (e.g. a green light) can be provided when target tissue is identified, and a negative feedback (e.g. a red light) can be provided if non-target tissue is identified (e.g. identified as tissue proximate an area of treatment energy 500 or other treatment delivery). User interface 250 can provide labels (e.g. text or icons) representing certain types of tissue, such as according to specific tissue type or a more general tissue category. User interface 250 can combine TCI 400 with a location tracking to a two and/or three-dimensional spatial map tissues in and/or at least proximate the surgical site.

In some embodiments, probe 100 can include two or more pairs of probe electrodes 151 configured to deliver bipolar energy (e.g. deliver bipolar signals) such as to allow measurement of larger areas of tissue. In some embodiments, the area of tissue receiving the bipolar energy (e.g. a bipolar delivery signal) is at least 0.1 cm² and/or no more than 10 cm². For example, probe 100 can comprise a two-dimensional array of probe electrodes 151, such that the array can be placed over an area of an organ and/or other tissue area. As discussed hereabove, the array of probe electrodes 151 can be formed from printed circuit board or flex circuit technology. With each adjacent pair of probe electrodes 151 in the electrode array providing a signal of a characteristic of tissue proximate that pair, an entire tissue area can be characterized. Data from TCI 400 can be combined with known anatomical data to display the tissue characteristics on an anatomical model of the tissue (e.g. each as displayed on user interface 250). In some embodiments, probe 100 comprises three or more electrodes 151 that are separated by unequal distances, such as to provide recorded signals 402 that represent volumes of tissue located at different distances from electrodes 151 (e.g. different depths below electrodes 151).

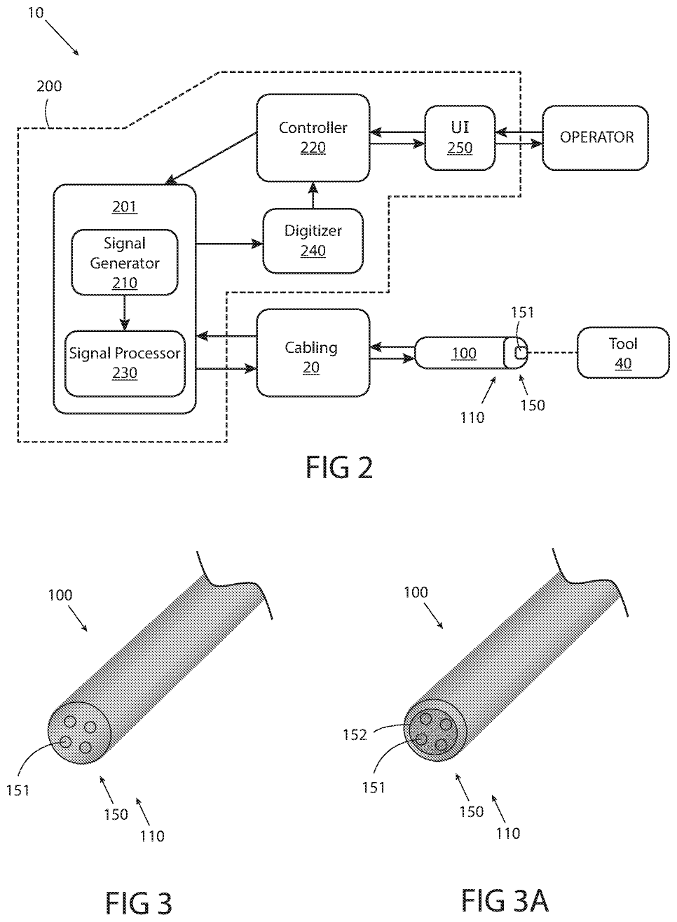

Referring now to FIG. 2, a system for characterizing tissue is illustrated, consistent with the present inventive concepts. System 10 includes a tissue probe 100 having a plurality of tissue-contacting electrodes, probe electrodes 151, such as for delivery of bipolar energy to tissue (e.g. delivery of bipolar-based drive signals 401 to tissue) and console 200. System 10 can be of similar construction and arrangement, and can include similar components to, system 10 as described hereabove in reference to FIG. 1. Probe electrodes 151 can be positioned at one or more locations of probe 100, such as at a distal portion, tip 110, of probe 100. A module 201 includes a signal generator 210, and one or more amplifiers, samplers, and/or other signal processing components, signal processor 230. Signal processor 230 can be configured to measure the amplitude and phase of signals recorded by probe electrodes 151 (e.g. at the location of tip 110). Signal generator 210 can be any known frequency generator such as a stepped frequency generator and/or a swept frequency generator. Module 201 is operably connected (e.g. at least electrically connected) with probe 100 and probe electrodes 151 via cable 20, which can include one or more wires and/or other conduits. In some embodiments, cable 20 comprises noise canceling cabling, noise-shielded cabling and/or otherwise noise-reducing cabling. In some embodiments, cable 20 comprises one or more optical fibers, fluid delivery tubes, waveguides, and/or other sold or hollow filaments. Module 201 (e.g. signal generator 210) selectively applies a signal to probe 100 when the probe is located at the surgical site and in contact with and/or at least in close proximity to tissue (e.g. neighboring tissue). A processor or micro-controller, controller 220, receives a signal resulting from transmission of the applied signal through tissue by probe 100. Controller 220 receives the signal from the tissue transmitted by probe 100 and cable 20, through module 201 and an analog to digital converter, digitizer 240, and measures a change in the signal resulting from transmission of the energy though the tissue.

The signal resulting from transmission of the drive signals 401 through neighboring tissue can be measured in terms of a state and/or change (relative change) in one or more of voltage, power, energy, current, phase and/or frequency. System 10 and controller 220 can use one or more of these measurements, and/or a calculated quantity such as electrical impedance or resistance of the tissue to produce TCI 400. A clinician or other operator of system 10 receives TCI 400 and/or other information from one or more feedback indicators or other user interfaces, UI 250 shown, which can include information about the tissue proximate probe electrodes 151, such as information for use in making decisions during a surgery or other clinical procedure. With this tissue information provided by system 10, the surgeon or other clinician can make safer and/or faster decisions during a clinical procedure.

System 10 can include tool 40, which can comprise a calibration load used to calibrate system 10 as described hereabove in reference to FIG. 1.

Referring additionally to FIG. 3, tip 110 of probe 100 is illustrated, consistent with the present inventive concepts. Probe 100 of FIG. 3 can be of similar construction and arrangement as probe 100 as described hereabove in reference to FIG. 1. In some embodiments, probe 100 includes a detachable tip 110 (e.g. a disposable tip) positioned at a distal end of probe 100. Tip 100 can include a plurality of probe electrodes 151. In some embodiments, tip 110 is replaced after each use (e.g. tip 100 is disposed of after each clinical procedure on a single patient, and a replacement tip 110 is positioned on probe 100 for a subsequent procedure). Alternatively, probe 100 (in its entirety) can be replaced after each clinical procedure. In the example of FIG. 3, a distal end of the probe 100 is shown with a curved or hemispherical tip 110 which includes a matrix or other array of a plurality of probe electrodes 151, electrode assembly 150. Although a spherically shaped tip 110 is shown, other shapes of tip surfaces, such as ellipsoidal or other atraumatic shapes can also be used. An atraumatic shape of the tip 110 allows the probe electrodes 151 to smoothly travel through and over tissue within the surgical site without “catching” the tissue. In some embodiments, two probe electrodes 151 are used for bipolar transmission of the signal through a localized tissue section. In some embodiments, a double row of electrodes (e.g. a 1 by 2, or 2 by 2 array) is located across tip 110, such as to allow characterization of a larger section of tissue as probe 100 is advanced across the tissue surface.

Referring additionally to FIG. 3A, a perspective view of the distal portion of probe 100 including one or more covered probe electrodes 151 is illustrated, consistent with the present inventive concepts. Probe 100 of FIG. 3A can be of similar construction and arrangement as probe 100 described hereabove in reference to FIG. 1. In the embodiment of FIG. 3A, probe electrodes 151 positioned on tip 110 are covered with a housing and/or a coating, coating 152. Coating 152 can comprise a polymer, such as polyethylene, which encloses probe electrodes 151 and allows probe electrodes 151 to be in close proximity to, but not in contact with, tissue, as probe 100 (e.g. coating 152) is brought in contact with tissue. This non-contacting design is particularly useful for fluid containing environments where a significant amount of the electrical signal will tend to pass in the fluid between probe electrodes 151, instead of passing through the tissue. Tip 110 can include an atraumatic (e.g. smooth) shape for contacting tissue intra-operatively, such as is described hereabove in reference to FIG. 3.

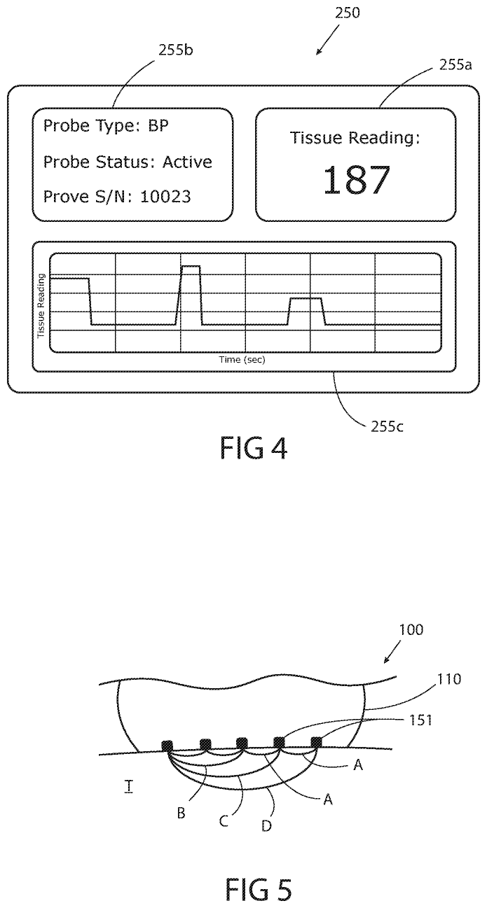

Referring now to FIG. 4, a front view of a user interface is shown, consistent with the present inventive concepts. User interface 250 includes a numerical tissue reading 255a, probe information 255b including probe type and probe status, and a graphical output 255c, such as tissue type measurements displayed over time. Many other possible user interface configurations can be used either as stand-alone displays or incorporated into other displays already present in the room in which the clinical procedure takes place (e.g. an operating room). User interface 250 of FIG. 4 can be of similar construction and arrangement as user interface 250 described hereabove in reference to FIG. 1.

Referring now to FIG. 5, a schematic cross section of a probe tip including an array of electrodes is illustrated, consistent with the present inventive concepts. Probe 100 includes a distal portion, tip 110, and multiple probe electrodes 151 positioned on the distal end of tip110, all as shown. Probe 100 of FIG. 5 can be of similar construction and arrangement as probe 100 as described hereabove in reference to FIGS. 1, 2, 3 and/or 3A. Lines A-D illustrate the varying depths of signal penetration and locations of tissue information (e.g. TCI 400) which can be achieved with multiple probe electrodes 151, by using multiple combinations of the same set of probe electrodes 151 (e.g. by delivering drive signals 401 through the multiple combinations of the probe electrodes 151). Although FIG. 5 schematically shows the path of the signal through the tissue T in cross section, the use of a 2D electrode array can achieve additional information about tissue T at multiple locations and depths simultaneously, such as to produce an anatomical map of tissue characteristics for tissue T. Tip 110 can be used for a point check at a single tissue location, or for a check of multiple tissue locations such as by dragging tip 110 along the surface of tissue T.

As described herein, TCI 400 provided by probe 100 and system 10 can identify or characterize structures (e.g. tissue) of interest and assist a surgeon or other clinician in locating an appropriate location to start and/or end a tissue diagnosis and/or treatment (e.g. delivery of treatment energy 500 for tissue cutting. ablating, or other treatment). For example, TCI 400 comprising nerve identification can help a surgeon locate an area to cut which provides a path between nerves (e.g. avoiding each nerve).

Referring now to FIG. 6, a time-domain representation of a waveform comprising a time-interleaved measurement signal and treatment energy delivery signal is illustrated, consistent with the present inventive concepts. The vertical axis represents the amplitude of a delivered interleaved waveform, while the horizontal axis represents time. The treatment energy 500 delivered (e.g. electrosurgical energy) is represented by the large amplitude waveforms, which includes an off-time period, labeled as PoFF. During this off-time duration, drive signals 401 can be delivered to neighboring tissue, and the recorded signals 402 recorded. The recorded signals 402 can be used by system 10 to produce TCI 400 (e.g. tissue characteristics of the neighboring tissue). This interleaved energy delivery provides simplified use for an operator of system 10, as described hereabove. In some embodiments, separation of the treatment energy 500 delivery, and the drive signal 401 delivery, enables a lower-cost design for console 200.

Referring now to FIG. 7, a schematic view of a probe further configured as a monopolar treatment energy delivery device is illustrated, consistent with the present inventive concepts. Probe 100 includes two or more probe electrodes 151, such as the three shown, such that probe electrode 151a is positioned in a central location, and probe electrodes 151b and 151c are positioned alongside probe electrode 151a. Probe electrodes 151a-c are electrically connected to signal generator 210 via cable 20 and isolation network 280, isolation network 280 including switches 281a and 281b, connected as shown. Signal generator 210 comprises a drive signal generator portion 210a (e.g. a low voltage or otherwise low energy drive signal generator), and a treatment energy generator portion 210b (e.g. a high voltage or otherwise high energy treatment signal generator) that provides treatment energy 500 to probe 100. Isolation network 280 is configured to electrically connect generator portions 210a and 210b as appropriate, as well as to protect the various electronic components of console 200 (e.g. protect the tissue characterization circuitry of controller 220) and protect the patient and operator(s) of system 10 (e.g. from electrical shock). One or more wires can be configured to connect probe electrode 151a to console 200, and they can be configured to transmit wideband signals. The one or more wires can be configured to connect electrodes 151b-c to console 200, can they can be configured to only carry the treatment energy 500 signal (typically less than 2 Mhz).

When probe 100 is being used in a treatment energy 500 delivery mode, switch 281a is closed, electrically connecting treatment signal generator portion 210b to center probe electrode 151a, and switch 281b is also closed, electrically connecting probe electrodes 151b and 151c to center probe electrode 151a (e.g. further connecting probe electrodes 151b-c to generator portion 210b). In some embodiments, isolation network 280 further includes switch 281c, a double pole double throw switch which can connect probe electrode 151a to one side of bipolar drive signal delivery of generator portion 210a and probe electrodes 151b-c to the other side of bipolar drive signal delivery of generator portion 210a. Switch 281c is open during treatment energy 500 delivery mode, isolating generator portion 210a from the treatment energy 500 delivered by generator portion 210b. Treatment energy 500 is delivered in parallel (and simultaneously) via probe electrodes 151a-c, with electrode 30 configured as a return path for treatment energy 500 delivered by all three of probe electrodes 151a-c (e.g. electrode 30 comprises a ground patch electrode positioned on the patient's back and/or leg for monopolar-based treatment energy 500 delivery). In some embodiments, probe electrode 151c is not included, and treatment energy 500 is delivered only by probe electrodes 151a-b.

When in a tissue characterization mode (e.g. during an “off period” of treatment energy 500 delivery), switches 281a and 281b are opened, and switch 281c (if present) is closed. In this state, probe electrodes 151b and 151c (if included) can be configured as return paths for drive signals 401 delivered via probe electrode 151a. Recorded signals 402 are received and processed by controller 220 to produce TCI 400, such as is described hereabove in reference to FIG. 1.

Referring now to FIG. 8, a schematic view of a probe further configured as a bipolar-based treatment energy delivery device is illustrated, consistent with the present inventive concepts. Probe 100 includes (at least) two or more probe electrodes, two shown, 151a and 151b positioned in side-by-side arrangement. Probe electrodes 151a-b are electrically connected to signal generator 210 via cable 20 and isolation network 280, isolation network 280 including switches 282a-e, connected as shown. Signal generator 210 comprises a drive signal generator portion 210a (e.g. a low voltage or otherwise low energy signal generator), and a treatment signal generator portion 210b (e.g. a high voltage or otherwise high energy signal generator) that provides treatment energy 500 to probe 100. Isolation network 280 is configured to electrically connect generator portions 210a and 210b as appropriate, as well as to protect the various electronic components of console 200 (e.g. protect the tissue characterization circuitry of controller 220). The one or more wires can be configured to connect probe electrodes 151a-b to console 200, and can be configured to transmit wideband signals.

When probe 100 is being used in a treatment energy 500 delivery mode, switches 282d and 282e are closed, electrically connecting treatment energy generator portion 210b to probe electrodes 151b and 151a respectively. Switches 282a-c are all open. Treatment energy 500 is delivered in a bipolar mode via probe electrodes 151a-b.

When transitioning to a tissue characterization mode (e.g. during an “off period” of treatment energy 500 delivery), switches 282d-e are opened, switches 282a-b remain open, and switch 282c is momentarily closed (e.g. to discharge one or more components of system 10 such as to discharge the cable capacitance of cable 20).

During the tissue characterization mode (e.g. also during an “off period” of treatment energy 500 delivery), switches 282d-e remain open, switch 282c is opened, and switches 282a-b are closed. In this state, probe electrodes 151a-b deliver bipolar tissue characterization signals. Recorded signals 402 are received and processed by controller 220 to produce TCI 400, such as is described hereabove in reference to FIG. 1.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for characterizing tissue comprising:
a tissue probe comprising a plurality of probe electrodes;
a signal generator in communication with the probe electrodes for delivering a drive signal to at least one probe electrode;
a controller for receiving a recorded signal from one or more probe electrodes, the recorded signal resulting from delivery of the drive signal through tissue proximate the plurality of probe electrodes,
wherein the controller is configured to determine patient-to-patient differences when the tissue probe is proximate a known type of patient tissue and/or bodily fluid, and
produce tissue characterization information based on the recorded signal and the patient-to-patient differences; and
a user interface for providing the tissue characterization information to an operator of the system.

2.-42. (canceled)
